(19) United States
(12) Patent Application Publication
(10) Pub. No.: US 2020/0273570 A1
(43) Pub. Date: Aug. 27, 2020

(54) PREDICTIVE ANALYSIS PLATFORM

(71) Applicant: Accenture Global Solutions Limited, Dublin (IE)

(72) Inventors: Revathi SUBRAMANIAN, San Diego, CA (US); Hongrui GONG, San Diego, CA (US); Lingjun A. HE, San Diego, CA (US); Stewart DE SOTO, Escondido, CA (US); Aaron Goodman LEVINE, Encinitas, CA (US)

(21) Appl. No.: 16/282,905

(22) Filed: Feb. 22, 2019

(51) Int. Cl. G16H 50/20; G16H 15/00; G16H 10/60; G06N 5/04; G06N 20/00

(52) U.S. Cl. CPC G16H 50/20; G16H 15/00; G06N 20/00; G06N 5/04; G16H 10/60

(57) ABSTRACT

A device may receive, from multiple systems, data related to an individual. The device may anonymize, after receiving the data and using an anonymization technique, information included in the data that identifies the individual. The device may apply a formatting to the data after anonymizing the information that identifies the individual. The device may identify, after applying the formatting to the data, historical data related to the individual, to a provider associated with a claim for care, or to historical claims, and population data associated with demographics of the individual. The device may process, in association with identifying the historical data and the population data, the data using a machine learning model. The machine learning model may be associated with generating a prediction related to the individual or the care provided to the individual. The device may perform one or more actions based on the prediction.

### BACKGROUND

A computer system is a combination of hardware and software. A computer system stores data and/or uses the data. Different systems may store different types of data and may use the data for different purposes.

### SUMMARY

According to some implementations, a method may comprise: receiving, by a device and from multiple systems, data related to an individual, wherein the data includes claim data related to a claim for care provided to the individual, demographic data related to demographics of the individual, and provider data related to a provider associated with the care; detecting, by the device, a type of the data after receiving the data, wherein the type of the data includes at least one of an image type or a text type; processing, by the device, the data based on the type of the data using at least one of: an image processing technique for the image type, or a text processing technique for the text type; applying, by the device, a formatting to the data after processing the data based on the type of the data using the at least one of the image processing technique or the text processing technique; identifying, by the device and after applying the formatting to the data, historical data related to the individual, to the provider associated with the claim for the care, or to historical claims with a similar diagnosis or procedure code as the claim, and population data associated with the demographics of the individual; processing, by the device, the identified historical data and population data, using a machine learning model, wherein the machine learning model generates a prediction related to the care for the individual or a value of the care for the individual; and performing, by the device, one or more actions based on the prediction.

According to some implementations, a device may comprise: one or more memories; and one or more processors, communicatively coupled to the one or more memories, to: receive, from multiple systems, data related to an individual, wherein the data includes claim data related to a claim for care provided to the individual, demographic data related to demographics of the individual, and provider data related to a provider associated with the care; detect a type of the data after receiving the data, wherein the type of the data includes at least one of an image type or a text type; process the data based on the type of the data using at least one of: an image processing technique for the image type, or a text processing technique for the text type; identify, after processing the data based on the type of the data, historical data related to the individual, to the provider associated with the care, or to historical claims with a similar diagnosis or procedure code as the claim, and population data related to the demographics of the individual; process, in association with identifying the historical data and the population data, the data using a machine learning model, wherein the machine learning model is associated with generating a prediction related to the individual or the care for the individual; and perform one or more actions based on the prediction.

According to some implementations, a non-transitory computer-readable medium may store instructions, the instructions comprising: one or more instructions that, when executed by one or more processors of a device, cause the one or more processors to: receive, from multiple systems, data related to an individual, wherein the data includes claim data related to a claim for care provided to the individual, demographic data related to demographics of the individual, and provider data related to a provider associated with the care; anonymize, after receiving the data and using an anonymization technique, information included in the data that identifies the individual; apply a formatting to the data after anonymizing the information that identifies the individual; identify, after applying the formatting to the data, historical data related to the individual to the provider associated with the claim for the care, or to historical claims with a similar diagnosis or procedure code as the claim, and population data associated with the demographics of the individual; process, in association with identifying the historical data and the population data, the data using a machine learning model, wherein the machine learning model is associated with generating a prediction related to the individual or the care provided to the individual; and perform one or more actions based on the prediction.

### BRIEF DESCRIPTION OF THE DRAWINGS

FIGS. 1-2K are diagrams of example implementations described herein.

FIG. 3 is a diagram of an example environment in which systems and/or methods described herein may be implemented.

FIG. 4 is a diagram of example components of one or more devices of FIG. 3.

FIGS. 5-7 are flow charts of example processes for performing predictive analysis.

### DETAILED DESCRIPTION

The following detailed description of example implementations refers to the accompanying drawings. The same reference numbers in different drawings may identify the same or similar elements.

Various entities associated with providing care to an individual store data across separate, isolated systems. For example, the various entities may store data related to historical care provided to individuals, data related to demographics of the individuals, and/or the like. The isolation and/or separation prevents the systems from communicating with each other, such as to share the data, to analyze data from different systems, and/or the like. In addition, even if different systems were capable of communicating with each other, different formatting used with data in different systems, different levels of anonymization and/or encryption, and/or the like would prevent the different systems for using each other's data. For example, a first system may not be capable of using data, from a second system, related to historical care for an individual to analyze the data in a context of a larger population, in a context of other individuals with the same demographics as the individual, and/or the like due to differences in types of data, formatting of data, anonymization, and/or the like between the first system and the second system.

Some implementations described herein provide a predictive analysis platform that is capable of processing data, from multiple separate and isolated systems, related to care provided to multiple individuals, claims for care provided to the multiple individuals, and/or the like to apply a uniform formatting to the data, to transform the data from one type to another type, and/or the like. In addition, the predictive analysis platform, based on applying the uniform formatting, transforming the data, and/or the like, may process the data from the multiple separate and isolated systems to perform various predictive analyses related to care provided to the multiple individuals. In this way, the predictive analysis platform can provide a standardized interface for data access between multiple systems associated with providing care to multiple individuals. In addition, the predictive analysis platform may utilize machine learning models that have been trained on anonymized data to perform the various predictive analyses, thereby facilitating an analyses of data related to care provided to an individual without providing a user of the predictive analysis platform with access to the underlying data and without storing the underlying data (e.g., the predictive analysis platform may need to store the machine learning models, but not the data on which the machine learning models were trained). This improves a security and/or privacy of data that is accessible by the predictive analysis platform. Further, by utilizing data that has a uniform formatting, data that has been transformed to a particular type of data, and/or the like, the predictive analysis platform utilizes fewer processing resources when processing data relative to attempting to process data with different formatting, data of different types, and/or the like.

In this way, several different stages of a process for predictive analysis improve speed and efficiency of the process and conserve computing resources (e.g., processor resources, memory resources, and/or the like). Furthermore, implementations described herein use a rigorous, computerized process to perform tasks or activities that were not previously performed.

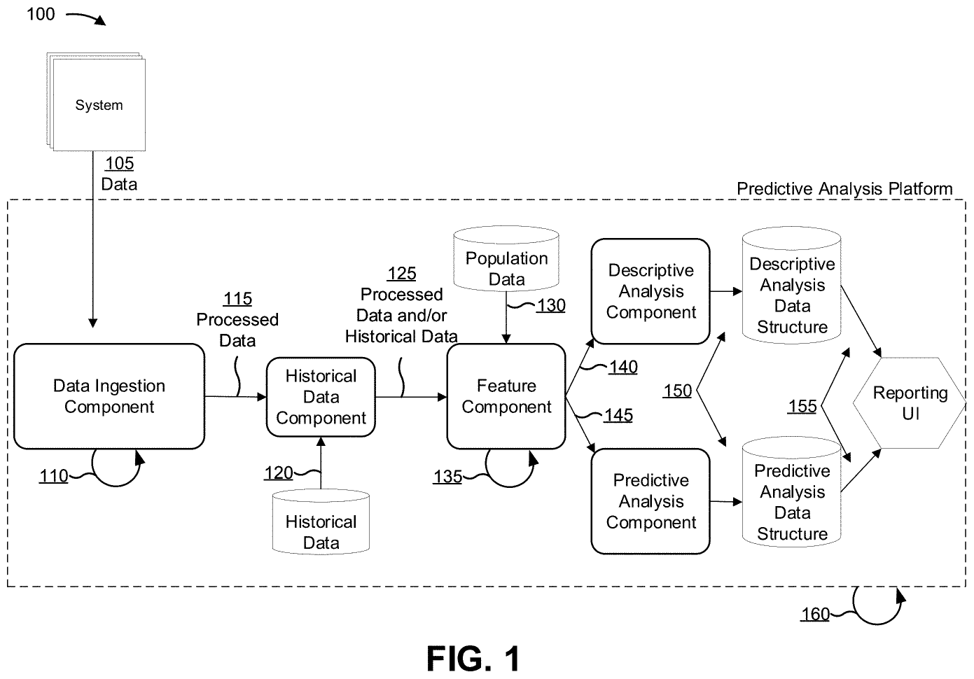

FIG. 1 is a diagram of one or more example implementations 100 described herein. As shown in FIG. 1, example implementation(s) 100 include various systems (e.g., a patient management system associated with a provider of care, a management system associated with a coverage entity, and/or the like) and a predictive analysis platform. The term “care” may refer to health-related activities performed by a provider of care (e.g., a licensed or unlicensed individual that performs the health-related activities for an individual (e.g., a patient), such as diagnosis, treatment, testing, imaging, rehabilitation, and/or the like). The term “coverage entity” includes an individual, an organization, a governmental entity, and/or the like that performs coverage-related activities for care provided to an individual, such as providing insurance coverage, reimbursement for care, coverage underwriting, and/or the like.

As shown by reference number 105, the various systems may provide data to the predictive analysis platform. For example, the various systems may provide data stored by the various systems, gathered by the various systems, generated by the various systems, input by users of the various systems, and/or the like. In some implementations, a system may provide the data in batch (e.g., may provide the data after storing and/or gathering an amount of data that satisfies a threshold), in real-time or near real-time (e.g., as the data is gathered and/or generated), periodically, according to a schedule, and/or the like. In some implementations, the predictive analysis platform may receive the data using a data ingestion component. For example, and as described elsewhere herein, the data ingestion component may pre-process the data after receiving the data to place the data in a form that the predictive analysis platform can use to perform other processing described herein.

In some implementations, the data may include claim data related to a claim for care provided to an individual. For example, the data may include information that identifies the individual, the care provided to the individual (e.g., a procedure code), a provider that provided the care (e.g., a name of the provider, a name of a practice of the provider, and/or the like), a care identifier that identifies types of care provided by the provider (e.g., terms like “dentist,” “pediatrician,” “physical therapist,” “masseuse,” and/or the like), a value (e.g., a cost, a reimbursement amount, a claimed amount, an amount paid, and/or the like) of the care provided to the individual, a location of the individual and/or the provider (e.g., an address of the individual and/or the provider), identifiers for specific care provided to the individual (e.g., a billing code), a type of a claim (e.g., a particular claim form used for the claim), a diagnosis associated with the claim (e.g., based on a diagnosis code included in the claim), and/or the like. Additionally, or alternatively, the data may include demographic data related to demographics of the individual. For example, the data may include information that identifies an age of the individual, a location of the individual, a gender of the individual, an ethnicity of the individual, an income level of the individual, and/or the like. Additionally, or alternatively, the data may include provider data related to a provider associated with care provided to an individual. For example, the data may include information that identifies a provider's specialty, a provider's location, a provider's facility affiliation, and/or the like. Additionally, or alternatively, the data may include historical data for historical claims, and the predictive analysis platform may aggregate and store the historical data by demographic, diagnosis, and/or the like.

In some implementations, the data may be anonymized (or partially anonymized). For example, the data may include anonymizing values for a name of an individual and/or a provider, for an address of the individual and/or the provider, for a telephone number of the individual and/or the provider, and/or the like. In some implementations, data from different systems may be anonymized in different manners. For example, different systems may use different anonymizing values and/or techniques. Use of different anonymizing values and/or techniques facilitates use of anonymized data by the predictive analysis platform, which improves a security and/or privacy of data accessible and/or used by the predictive analysis platform.

In some implementations, the data may be of a particular type. For example, the data may be of a text type, an image type, and/or the like. Continuing with the previous example, the predictive analysis platform may receive an image of a claim for care, may receive text of a claim for care, and/or the like. In some implementations, data from different systems may be of different types. In some implementations, the data may be formatted in a particular manner. For example, the data may have a formatting with regard to particular quantities of decimal places, such as for units of care provided (e.g., quantity of hours, units of medicine, and/or the like), acronyms used in the data, spaces between particular terms, and/or the like. In some implementations, data from different systems may have different formatting. In some implementations, the data may include various types of data elements. For example, data related to an individual may include data elements for a name of the individual, a location of the individual, a telephone number of the individual, and/or the like. In some implementations, data from different systems may include different combinations of data elements. For example, data for an individual from a first system may include data elements for a name of the individual and an address of the individual, but data for the individual from a second system may include data elements for the name of the individual, a city location of the individual, and a telephone number for the individual.

As shown by reference number 110, the data ingestion component of the predictive analysis platform may pre-process the data to form processed data. For example, the predictive analysis platform may pre-process the data using the data ingestion component after receiving the data, based on receiving input from a user of the predictive analysis platform to pre-process the data, after receiving an amount of the data that satisfies a threshold, after receiving the data from particular systems, and/or the like.

In some implementations, the data ingestion component may detect a type of the data in association with pre-processing the data. For example, the data ingestion component may detect a type of the data as an image type (e.g., an electronic document, a scan of a physical document, and/or the like), a text type, and/or the like. In some implementations, the data ingestion component may detect a type of the data based on a form of the data. For example, the data ingestion component may identify a form of the data from metadata associated with a file in which the data was provided to the predictive analysis platform, a type of the file, a source system that provided the data (e.g., a first system may provide text data and a second system may provide image data), and/or the like. As a specific example, the data ingestion component may detect the type of the data as a text type based on receiving the data in a text file (e.g., a comma-separated values (CSV) text file), in a spreadsheet file (e.g., where the data is in a tabular form of rows and columns) after performing a lookup of the form of the file in a data structure, based on metadata that indicates that the data is a text type, and/or the like. In some implementations, the data ingestion component may detect a type of the data based on a file extension of the data. For example, the data ingestion component may detect a file extension associated with a file in which the data was provided to the predictive analysis platform, and may perform a lookup of the file extension in a data structure to identify a corresponding type of the data.

In some implementations, the data ingestion component may process the data based on the type of the data (e.g., to extract the data from a file in which the data was received). For example, the data ingestion component may select a processing technique for the data based on the type of the data, prior to processing the data using the processing technique. As specific examples, the data ingestion component may select a text processing technique (e.g., a natural language processing technique, a text analysis technique, and/or the like) for a text type, an image processing technique (e.g., a computer vision technique, an optical character recognition (OCR) technique, a feature detection technique, and/or the like) for an image type, and/or the like. In some implementations, when processing the data using the processing technique, the data ingestion component may identify terms, phrases, symbols, numbers, and/or the like in the data.

In some implementations, the data ingestion component may apply a formatting to the data. For example, the data ingestion component may apply a formatting to the data after extracting the data from a file. In some implementations, when applying a formatting to the data, the data ingestion component may remove spaces from text, may convert data from an image to text, may convert text data to plain text, may expand an acronym and/or an abbreviation in the data to include complete terms and/or phrases, may contract a term and/or a phrase to an acronym and/or an abbreviation, may add or remove symbols from the data (e.g., may add or remove symbols such as “(,” “),” “-,” and/or the like from a telephone number), and/or the like. This conserves processing resources that would otherwise be consumed attempting to process differently formatted data.

In some implementations, the data ingestion component may anonymize the data. For example, the data ingestion component may anonymize the data after applying a formatting to the data, prior to applying the formatting, and/or the like. In some implementations, the data ingestion component may process particular data elements of the data (e.g., information that identifies an individual, or that could be used to identify an individual) using an anonymization technique to form anonymized identifiers. For example, the data ingestion component may process the data using data encryption (e.g., by processing values of a data element to form a random array of characters), character substitution (e.g., by replacing values of a data element with a particular value), character shuffling (e.g., by rearranging characters a value of a data element), number and/or date variance (e.g., by modifying numerical values by a predetermined amount, by modifying date values by a predetermined amount of time, and/or the like), nulling (e.g., by removing values for particular data elements), and/or the like to form an anonymized identifier and/or to anonymize the data. As specific examples, the data ingestion component may replace a name of an individual with a randomly generated array of alphanumeric characters and/or symbols, may remove values of a telephone number (or replace values of a telephone number with a character, a symbol, and/or the like) other than an area code of the telephone number, may anonymize an address in a similar manner so that only a street name, a zip code, and/or the like is not anonymized, and/or the like. In some implementations, the data ingestion component may anonymize the data prior to storing the data, using the data, providing the data for display, and/or the like. This facilitates maintaining of privacy of individuals associated with the data by reducing or eliminating a risk that unauthorized individuals will have access to non-anonymized data.

In some implementations, the data ingestion component may determine a signature of the data. For example, the data ingestion component may determine a signature of the data after anonymizing the data. In some implementations, a signature of the data may include information that identifies combinations of data elements, values for particular data elements, and/or the like associated with a record in the data. For example, for a record in claim data, the data ingestion component may determine that the data includes data elements for a name of the individual to which care was provided, a provider that provided the care, a location at which the care was provided, values for the previously mentioned data elements, and/or the like, and may determine a signature for claim data based on this combination of data elements, may determine a signature for claim data for a particular individual based on values for the data elements, and/or the like.

In some implementations, the data ingestion component may use a signature of the data to correlate anonymized data across different systems. For example, the data ingestion component may match a signature of data elements and/or values for the data elements from a first system to a similar combination of data elements and/or values in a second system, and may determine that the data from the first system is associated with the same individual based on the match. Additionally, or alternatively, and as another example, the predictive analysis platform may train a machine learning model (e.g., a natural language processing model) on signatures determined for the data, and the data ingestion component may identify the same data in different systems using the machine learning model (e.g., despite the same data in different systems including different combinations of data elements, different values for some of the data elements, and/or the like). As a specific example, and continuing with the previous examples, data from a first system for an individual may include different data elements than data from a second system for the individual (or a category of individual, such as a category based on a location of the individual, a demographic of the individual, and/or the like), and the data ingestion component may correlate the data across the two systems despite the data for the individual including different data elements in the two systems. This facilitates use of anonymized data across multiple systems in scenarios when the data ingestion component would not otherwise be capable of correlating data across multiple systems due to anonymized data, differences in data elements and/or values, and/or the like, thereby improving use of the data, conserving processing resources that would otherwise be consumed as a result of failing to correlate the data, and/or the like.

In some implementations, the predictive analysis platform may generate a machine learning model via training of the machine learning model, may receive a trained machine learning model (e.g., that another device has trained), and/or the like. For example, the predictive analysis platform may train the machine learning model to output a prediction related to future care to be provided to an individual, a value of future care to be provided to an individual (e.g., a cost, a reimbursement value, and/or the like), a likelihood that a claim associated with claim data is a legitimate claim (e.g., a likelihood that the claim is non-fraudulent), whether (and/or to what extent) particular demographic data has impacted a prediction, and/or the like, as described herein.

In some implementations, the predictive analysis platform may train the machine learning model on a training set of data. For example, the training set of data may include data related to historical claims and/or demographic data of individuals associated with the historical claims, and data that identifies historical patterns related to the historical claims and/or the demographic data. Additionally, or alternatively, when the predictive analysis platform inputs the data related to the historical claims, the demographic data, and/or the historical patterns into the machine learning model, the predictive analysis platform may input a first portion of the data as a training set of data (e.g., to train a machine learning model), a second portion of the data as a validation set of data (e.g., to evaluate an effectiveness of the training of the machine learning model and/or to identify needed modifications to the training of the machine learning model), and a third portion of the data as a test set of data (e.g., to evaluate a finalized machine learning model after training and adjustments to the training using the first portion of the data and the second portion of the data). In some implementations, the predictive analysis platform may perform multiple iterations of training of the machine learning model, depending on an outcome of testing of the machine learning model (e.g., by submitting different portions of the data as the training set of data, the validation set of data, and the test set of data).

In some implementations, when training the machine learning model, the predictive analysis platform may utilize a random forest classifier technique to train the machine learning model. For example, the predictive analysis platform may utilize a random forest classifier technique to construct multiple decision trees during training and may output a classification of data. Additionally, or alternatively, when training the machine learning model, the predictive analysis platform may utilize one or more gradient boosting techniques to generate the machine learning model. For example, the predictive analysis platform may utilize an xgboost classifier technique, a gradient boosting tree, and/or the like to generate a prediction model from a set of weak prediction models. In some implementations, the predictive analysis platform may utilize an isolation forest technique, or another type of machine learning technique, to train a machine learning model for fraud and/or anomaly detection.

In some implementations, when training the machine learning model, the predictive analysis platform may utilize logistic regression to train the machine learning model. For example, the predictive analysis platform may utilize a binary classification of the data related to the historical claims, the demographic data, and/or the historical patterns (e.g., whether the historical claims and/or the demographic data match the historical patterns) to train the machine learning model. Additionally, or alternatively, when training the machine learning model, the predictive analysis platform may utilize a Naive Bayes classifier to train the machine learning model. For example, the predictive analysis platform may utilize binary recursive partitioning to divide the data related to the historical claims, the demographic data, and/or the historical patterns into various binary categories (e.g., starting with whether the historical claims and/or the demographic data match the historical patterns). Based on using recursive partitioning, the predictive analysis platform may reduce utilization of computing resources relative to manual, linear sorting and analysis of data points, thereby enabling use of thousands, millions, or billions of data points to train a machine learning model, which may result in a more accurate machine learning model than using fewer data points.

Additionally, or alternatively, when training the machine learning model, the predictive analysis platform may utilize a support vector machine (SVM) classifier. For example, the predictive analysis platform may utilize a linear model to implement non-linear class boundaries, such as via a max margin hyperplane. Additionally, or alternatively, when utilizing the SVM classifier, the predictive analysis platform may utilize a binary classifier to perform a multi-class classification. Use of an SVM classifier may reduce or eliminate overfitting, may increase a robustness of the machine learning model to noise, and/or the like.

In some implementations, the predictive analysis platform may train the machine learning model using a supervised training procedure that includes receiving input to the machine learning model from a subject matter expert. In some implementations, the predictive analysis platform may use one or more other model training techniques, such as a neural network technique, a latent semantic indexing technique, and/or the like. For example, the predictive analysis platform may perform a multi-layer artificial neural network processing technique (e.g., using a two-layer feedforward neural network architecture, a three-layer feedforward neural network architecture, and/or the like) to perform pattern recognition with regard to patterns of historical claims and/or demographic data, patterns of historical claims and/or demographic data based on an accuracy of a historical predictions, and/or the like. In this case, using the artificial neural network processing technique may improve an accuracy of a supervised learning model generated by the predictive analysis platform by being more robust to noisy, imprecise, or incomplete data, and by enabling the predictive analysis platform to detect patterns and/or trends undetectable to human analysts or systems using less complex techniques.

As an example, the predictive analysis platform may use a supervised multi-label classification technique to train the machine learning model. For example, as a first step, the predictive analysis platform may map data associated with the historical claims, the demographics, and/or the historical patterns to a set of previously generated models after labeling the historical claims, the demographic data, and/or the historical patterns. In this case, the historical claims and/or the demographics may be characterized as having been accurately or inaccurately predicted, the historical patterns may be characterized as having been accurate or inaccurate, and/or the like (e.g., by a technician, thereby reducing processing relative to the predictive analysis platform being required to analyze each historical claim, demographic, and/or historical pattern). As a second step, the predictive analysis platform may determine classifier chains, whereby labels of target variables may be correlated (e.g., in this example, labels may be a result of a historical pattern and correlation may refer to historical patterns common to the different labels, and/or the like). In this case, the predictive analysis platform may use an output of a first label as an input for a second label (as well as one or more input features, which may be other data relating to the historical claims, the demographics, and/or the historical patterns), and may determine a likelihood that a particular historical claim is to be associated with at least one future claim based on a similarity to other historical claims that include similar data. In this way, the predictive analysis platform transforms classification from a multilabel-classification problem to multiple single-classification problems, thereby reducing processing utilization. As a third step, the predictive analysis platform may determine a Hamming Loss Metric relating to an accuracy of a label in performing a classification by using the validation set of the data (e.g., an accuracy with which a weighting is applied to each historical claim, demographic, and/or historical pattern and whether each historical claim and/or demographic is associated with a particular type and/or pattern of care, results in a correct historical pattern, and/or the like, thereby accounting for variations among historical claims and/or demographics). As a fourth step, the predictive analysis platform may finalize the machine learning model based on labels that satisfy a threshold accuracy associated with the Hamming Loss Metric, and may use the machine learning model for subsequent determination of other models.

As another example, the predictive analysis platform may determine, using a linear regression technique, that a threshold percentage of values of data elements, in a set of values of data elements, do not indicate future combinations of future care, whether a claim should be approved, and/or the like, and may determine that those values of data elements are to receive relatively low association scores. In contrast, the predictive analysis platform may determine that another threshold percentage of values of data elements does indicate future combinations of future care, whether a claim should be approved, and/or the like, and may assign a relatively high association score to those values of data elements. Based on the characteristics of the data elements indicating future combinations of care, whether a claim should be approved, and/or the like, or not, the predictive analysis platform may generate the model and may use the model for analyzing new data elements of claim data, demographic data, and/or the like that the predictive analysis platform identifies.

Accordingly, the predictive analysis platform may use any number of artificial intelligence techniques, machine learning techniques, deep learning techniques, and/or the like to determine future treatments for a diagnosis of an individual, to determine whether to approve a claim for care, and/or the like, as described herein.

In some implementations, the predictive analysis platform may generate a model and use the model to perform various processing described herein. For example, based on data relating to hundreds, thousands, millions or more entities across multiple systems, the predictive analysis platform may determine a combination of future care to be provided to an individual and/or a probability that different care will be provided to the individual. In this case, the model may be an item-based collaborative filtering model, a single value decomposition model, a hybrid recommendation model, and/or another type of model that enables various determinations described herein based on claim data, demographics data, and/or the like.

In some implementations, the predictive analysis platform may generate different machine learning models associated with generating different predictions, associated with processing data from different systems and/or of different forms, and/or the like. In some implementations, the predictive analysis platform may input data received from a system into a machine learning model (e.g., claim data, demographic data, population data, historical data, and/or the like), and the machine learning model may output information that identifies a predicted care that an individual may receive, a value of the predicted care, whether the predicted care matches that of other individuals with a similar diagnosis, similar demographics, and/or the like, and/or the like. In some implementations, the predictive analysis platform may use this information to generate a recommendation for care for an individual, to schedule the individual for the care, to predict a value for the care (e.g., to estimate a cost of the care), and/or the like, as described elsewhere herein.

As shown by reference number 115, the data ingestion component may provide processed data to a historical data component. For example, the predictive analysis platform may provide the processed data from the data ingestion component to the historical data component after the data ingestion component has pre-processed data from the various systems to form the processed data, based on receiving input from a user of the predictive analysis platform to provide the processed data from the data ingestion component to the historical data component, and/or the like. In some implementations, the predictive analysis platform may use the historical data component to gather historical data to be used as input to the machine learning model, to further train the machine learning model for a particular individual, provider, diagnosis, and/or the like, and/or the like.

As shown by reference number 120, the historical data component may identify historical data related to the individual, a category of the individual (e.g., a category based on demographic, location, diagnosis, and/or the like), related to a provider that provided care to the individual, related to historical claims with a similar diagnosis and/or procedure code as the claim, and/or the like. For example, the historical data component may identify the historical data in a data structure associated with the predictive analysis platform. In some implementations, the historical data may be related to historical claims associated with the individual, historical care provided to the individual, historical claims (for other individuals) associated with a provider that provided care to the individual, historical care provided to other individuals by the provider, and/or the like (e.g., based on the historical claims having a similar diagnosis as the claim (e.g., as identified in the historical claims), being associated with a similar procedure code as the claim, and/or the like). In some implementations, the historical data component may identify the historical data by performing a lookup of the historical data in the data structure, by querying the data structure, and/or the like. For example, the historical data component may perform a comparison of an anonymized identifier, generated when the data ingestion component anonymized the data, and multiple other anonymized identifiers stored in the data structure, and may identify the historical data based on a match (e.g., based on detecting a match). Additionally, or alternatively, and as another example, the historical data component may perform a comparison of a signature of processed data associated with an anonymized identifier to multiple signatures of other data stored in the data structure, and may identify the historical data based on a match of signatures. Additionally, or alternatively, and as another example, the historical data component may use a machine learning model to identify the historical data (e.g., by identifying historical data that has a similar signature to a signature of processed data associated with an anonymized identifier). For example, the historical data component may use a machine learning model to identify historical data as being associated with a same individual or provider as claim data based on the historical data and the claim data having similar, but different, combinations of data elements (e.g., which would cause the historical data and the claim data to have different signatures). This facilitates use of different sets of data that use different anonymized identifiers for a same individual, provider, and/or the like, thereby improving a use of the different sets of data.

As shown by reference number 125, the historical data component may provide the processed data and/or the historical data to a feature component. For example, the predictive analysis platform may provide the processed data and/or the historical data from the historical data component to the feature component after the historical data component has identified the historical data based on the processed data, based on receiving input from a user of the predictive analysis platform to provide the processed data and/or the historical data from the historical data component to the feature component, and/or the like.

As shown by reference number 130, the feature component may identify population data based on the demographic data associated with the individual. For example, the feature component may identify the population data in a data structure associated with the predictive analysis platform. In some implementations, the population may be related to historical claims, historical care, historical values of the historical claims and/or the historical care, and/or the like associated with individuals that have a similar combination of demographics as the individual, are associated with providers that are similar to the provider that provided the care to the individual, and/or the like. In some implementations, the feature component may identify the population data by performing a lookup of demographic data in the data structure, by querying the data structure using the demographic data as a set of parameters for a query, and/or the like, in a manner similar to that described herein. Additionally, or alternatively, the feature component may use a machine learning component to identify the population data. For example, the feature component may use the machine learning component to identify individuals in a data structure with similar demographics as the individual (e.g., a similar combination of demographics, such as a combination of a similar age, a same gender, a same geographic location, a similar income level, and/or the like), and may identify population data related to the individuals with the similar demographics.

As shown by reference number 135, the feature component may process the historical data and the population data using a machine learning model. For example, the feature component may process the historical data and the population data after identifying the historical data and/or the population data, based on receiving input from a user of the predictive analysis platform to process the historical data and/or the population data. In some implementations, the feature component may process patterns in the processed data, trends in the processed data, and/or the like in a context of the historical data and/or the population data.

In some implementations, the feature component may process the historical data and/or the population data in a context of the claim data, the demographic data, and/or the like for the individual, such as to generate a prediction related to the individual. For example, the feature component may process the historical data, the population data, the claim data, and/or the demographic data to generate a prediction related to future care to be provided to the individual. Continuing with the previous example, the feature component may generate a prediction that identifies future care to be provided to the individual, a timing for the future care, whether the care and/or the future care matches a diagnosis identified in the claim data, and/or the like.

Additionally, or alternatively, and as another example, the feature component may generate a prediction related to a value of the care and/or the future care. Continuing with the previous example, the feature component may determine a predicted cost of the future care, whether an amount to be reimbursed for the care matches the provider's history (or a history for other providers), and/or the like. Additionally, or alternatively, and as another example, the feature component may generate a prediction related to a diagnosis. For example, the feature component may generate a prediction related to whether a diagnosis matches the care identified in the claim data, a change in the diagnosis in the future, an accuracy of the diagnosis, a value of the diagnosis over period of time, and/or the like.

Additionally, or alternatively, and as another example, the feature component may generate a prediction related to whether a claim is a legitimate claim. Continuing with the previous example, the feature component may determine whether a claim associated with the claim data is a fraudulent claim, was submitted by mistake, and/or the like (e.g., based on a pattern of the claim data associated with the claim in a context of the historical data, the population data, and/or the like) using the machine learning model that was trained in the manner described elsewhere herein. Additionally, or alternatively, and as another example, the feature component may generate a prediction related to whether a claim is abnormal for the individual, the provider, a combination of demographics, and/or the like.

In some implementations, the feature component may generate a score in association with generating a prediction. For example, a machine learning model that the feature component uses may output a score in association with outputting a prediction. In some implementations, the score may indicate a similarity between the processed data received from the various systems and the historical data and/or the population data. For example, the score may indicate a degree to which the processed data matches a pattern of values in the historical data and/or the population data. Continuing with the previous example, the feature component may generate a prediction based on the score (e.g., a prediction that the claim is a legitimate claim, that a value of care will match historical values for historical care, and/or the like). Additionally, or alternatively, the score may indicate a confidence level for a prediction. For example, the score may indicate a confidence level (e.g., a high confidence, a medium confidence, or a low confidence) based on a degree to which patterns of the processed data match patterns of the historical data and/or the population data.

As shown by reference numbers 140 and 145, the feature component may provide a prediction, claim data, demographic data, historical data, and/or population data to a descriptive analysis component and/or a predictive analysis component. For example, the feature component may provide claim data, demographic data, historical data, and/or population data to the descriptive analysis component and may provide a prediction to the predictive analysis component.

In some implementations, the descriptive analysis component may process claim data, demographic data, historical data, and/or population data to perform an analysis related to the claim data, the demographic data, the historical data, and/or the population data (e.g., may perform an analysis in a context of the claim data, the demographic data, the historical data, and/or the population data). For example, the descriptive analysis component may perform an analysis of a value of care provided to an individual relative to a value for historical care provided to other individuals with a same diagnosis, a similar combination of demographics, a same provider, and/or the like, may perform an analysis of a value of the care over time (e.g., a trend in the value, a pattern in the value, and/or the like), and/or the like. Additionally, or alternatively, and as another example, the descriptive analysis component may perform an analysis of care, such as over time for the individual (e.g., may identify a trend and/or a pattern in care-related activities for the individual over time), by demographics (e.g., may determine whether a combination of care-related activities matches other individuals with a similar combination of demographics), and/or the like.

In some implementations, the predictive analysis component may process a prediction to perform an analysis of the prediction (e.g., in a context of the claim data, the demographic data, the historical data, and/or the population data). For example, the predictive analysis component may perform a comparison of predicted values related to care and historical values related to historical care (e.g., to determine a difference between the predicted values and the historical values, whether a pattern and/or a trend in the predicted values matches a historical pattern and/or a historical trend in the historical values, and/or the like). Additionally, or alternatively, and as another example, the predictive analysis component may perform a comparison of a combination of care-related activities predicted to be provided to an individual and historical combinations of care-related activities provided to other individuals with a same diagnosis, with a same provider, with a similar combination of demographics, and/or the like. For example, the predictive analysis platform may determine whether the combination of care-related activities matches historical combinations of care-related activities. Additionally, or alternatively, and as another example, the descriptive analysis component may determine whether a predicted length of care for the individual matches historical length of care for other individuals with a same diagnosis, with a same provider, with a similar combination of demographics, and/or the like.

In some implementations, the predictive analysis platform (e.g., using the descriptive analysis component and/or the predictive analysis component) may perform various other analyses of predictions, claim data, demographic data, historical data, population data, and/or the like. For example, the predictive analysis platform may perform an analysis of whether the claim associated with the claim data is a legitimate claim. Continuing with the previous example, the predictive analysis platform may determine whether a claim is a fraudulent claim based on a degree to which the claim data matches historical data and/or population data for demographics of an individual. Additionally, or alternatively, and as another example, the predictive analysis platform may perform an analysis of whether a coverage entity should provide coverage to an individual. Continuing with the previous example, the predictive analysis platform may perform an analysis of predicted care, a value of the predicted care, and/or the like for an individual, and may determine to approve or deny the individual for coverage (e.g., for insurance coverage based on the predicted care being different than an expected care for a diagnosis, based on a value of the predicted care, and/or the like).

As specific examples of analyses, the descriptive analysis component and/or the predictive analysis component may perform a prediction related to care to be provided to an individual (e.g., a prediction of a service bundle of care to be provided for a given diagnosis), a cost of the care to be provided (including procedure costs, service bundle costs, and/or the like). Additionally, or alternatively, the descriptive analysis component and/or the predictive analysis component may perform a gap analysis of patterns of care to be provided to different individuals with a similar diagnoses, with the same or different demographics, and/or the like (e.g., to identify differences among care to be provided to different individuals). In this case, the predictive analysis platform may analyze (e.g., assess and/or quantify) a gap in services provided to, and the cost across, different types of individuals, and may provide a result of this analysis for display, in a report, and/or the like (e.g., in a summarized format that identifies various statistics related to different demographic characteristics). In some implementations, the predictive analysis platform may identify best practices for care provided to individuals by identifying optimal care-value combinations provided to individuals with a particular diagnosis, and identifying gaps in care among different demographic profiles. In some implementations, the predictive analysis platform may generate recommendations (e.g., policy recommendations) for improving a quality of care provided to individuals (e.g., based on a result of a gap analysis) while maximizing value of the care across a demographic.

In some implementations, the predictive analysis platform (e.g., using the descriptive analysis component and/or the predictive analysis component) may generate a score for a result of an analysis. For example, the predictive analysis platform may use a machine learning model to perform an analysis, and the machine learning model may output a score in association with outputting a result of an analysis. In some implementations, the score may indicate a confidence level for a result of an analysis. For example, the machine learning model may output a score based on a degree to which processed data processed during the analysis matches data on which the machine learning model was trained (e.g., a relatively better match between the processed data and the data on which the machine learning model is trained may result in a score associated with a relatively higher confidence level). Additionally, or alternatively, and as another example, the machine learning model may output a score based on a degree to which historical results of historical analyses have been accurate. Continuing with the previous example, the predictive analysis platform may monitor data related to prior analyses over time to determine whether the historical analyses were accurate, and may generate a score for a new analysis based on an accuracy of the historical analyses. Additionally, or alternatively, and as another example, the score may indicate a likelihood that predicted care (e.g., a service bundle, treatment, and/or the like) is related to a diagnosis associated with a claim.

In some implementations, the predictive analysis platform (e.g., using the descriptive analysis component and/or the predictive analysis component) may perform a scenario analysis with regard to a prediction. For example, the predictive analysis platform may determine a manner in which a prediction, a score, a result of an analysis, and/or the like may change with different processed data by simulating changes in processed data on which the prediction, the score, and/or the like are based (e.g., by modifying values of the processed data). In some implementations, the predictive analysis platform may perform a value analysis for care. For example, the predictive analysis platform may analyze a cost of an individual procedure, a service bundle, a lifetime of care, and/or the like for a given diagnosis (e.g., whether the cost matches a historical cost, satisfies a threshold, and/or the like). In some implementations, the predictive analysis platform may generate a recommendation based on a result of the scenario analysis. For example, a particular scenario (e.g., a different provider, a different combination of care, and/or the like) may be associated with an improved score, and the predictive analysis platform may generate a recommendation to implement changes to a current scenario to match the particular scenario.

As shown by reference number 150, the descriptive analysis component and the predictive analysis component may store results of performing various analyses and/or processed data used to perform the various analyses in various data structures. For example, the descriptive analysis component may store processed data and/or results of performing various analyses in a descriptive analysis data structure and the predictive analysis component may store processed data and/or results of performing various analyses in a predictive analysis data structure. As shown by reference number 155, the predictive analysis platform may use a reporting user interface (UI) to provide processed data, results of analyses, predictions, and/or the like for display. For example, the predictive analysis platform (e.g., using the descriptive analysis component and/or the predictive analysis component) may access the processed data, the results of the analyses, the predictions, and/or the like in the various data structures, and may populate various UIs with the processed data, the results, the predictions, and/or the like. In some implementations, the predictive analysis platform may update the UIs in real-time, near real-time, periodically, according to a schedule, and/or the like.

As shown by reference number 160, the predictive analysis platform may perform one or more actions. For example, the predictive analysis platform may perform the one or more actions after processing the historical data and the population data using a machine learning model, based on input from a user of the predictive analysis platform, based on an interaction of a user of the predictive analysis platform with a UI, and/or the like.

In some implementations, the predictive analysis platform may generate a report related to a prediction that the predictive analysis platform generated, an analysis that the predictive analysis platform performed, and/or the like, and may output the report for display. Additionally, or alternatively, the predictive analysis platform may cause a claim to be approved or denied based on performing an analysis related to the claim, as described herein. For example, the predictive analysis platform may configure a value in a data structure that indicates that the claim is to be approved or denied and/or that the claim is to be further reviewed by an individual, and may send a message to a client device (e.g., the message may include information that indicates that the claim is to be approved or denied). Additionally, or alternatively, the predictive analysis platform may cause an individual to be approved or denied for coverage by a coverage entity based on a result of an analysis in a manner that is the same as or similar to that described with regard to approving or denying a claim. Additionally, or alternatively, the predictive analysis platform may cause a value for a claim to be adjusted based on a result of an analysis. For example, if a value of the care associated with a claim does not match a value of care for other similar claims (e.g., for other similar diagnoses), the predictive analysis platform may send a set of instructions to a device to adjust the value of the claim.

Additionally, or alternatively, the predictive analysis platform may send a message to a client device associated with a provider, a case worker, and/or the like. For example, the predictive analysis platform may send a message to a client device that identifies a result of an analysis performed by the predictive analysis platform (e.g., an analysis of care provided to or predicted to be provided to an individual, an analysis of a diagnosis, and/or the like). Additionally, or alternatively, the predictive analysis platform may schedule care for the individual based on a prediction, an analysis, and/or the like. For example, the predictive analysis platform may generate calendar items on electronic calendars associated with a provider and/or an individual to schedule the provider and/or the individual for the care based on care predicted to be provided to the individual by the provider. Additionally, or alternatively, the predictive analysis platform may send a set of instructions to a device associated with providing care to an individual to cause the device to be scheduled to provide care to the individual at a particular time, to cause the device to provide care to the individual, and/or the like.

In this way, a predictive analysis platform facilitates use of data from different systems with different formatting, of different types, with different levels and/or types of anonymization, and/or the like, such as to analyze the data, to generate a prediction related to the data, and/or the like. This conserves computing resources that would otherwise be consumed attempting to use data from different systems with different formatting, of different types, with different levels and/or types of anonymization, and/or the like. In addition, some implementations described herein apply a uniform formatting to data, transform the data to a common type of data, and/or the like, thereby improving a form of the data for use in the manner described herein (e.g., which conserves memory resources, processing resources, and/or the like via the improved form). Further, some implementations described herein facilitate performance of these operations with anonymized data. This maintains a privacy of individuals associated with the data, reduces or eliminates unauthorized access to portions of the data that can identify an individual associated with the data, and/or the like.

As indicated above, FIG. 1 is provided merely as one or more examples. Other examples may differ from what is described with regard to FIG. 1.

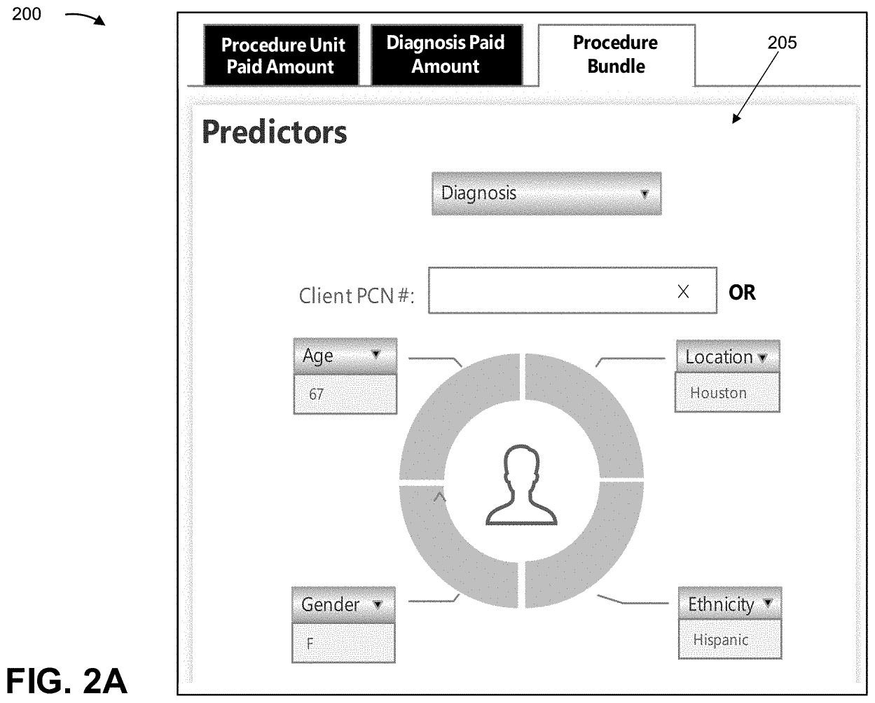

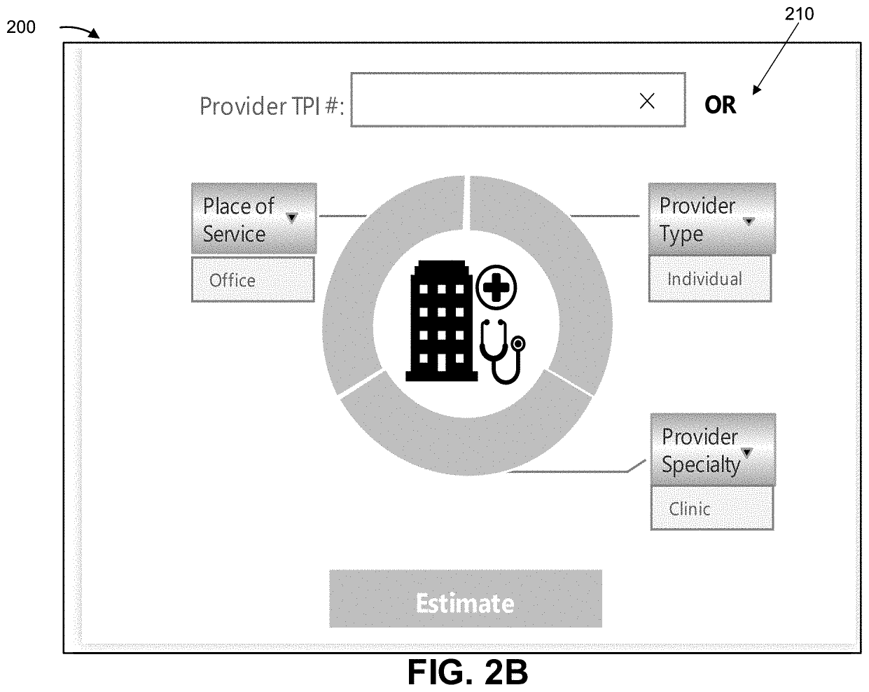

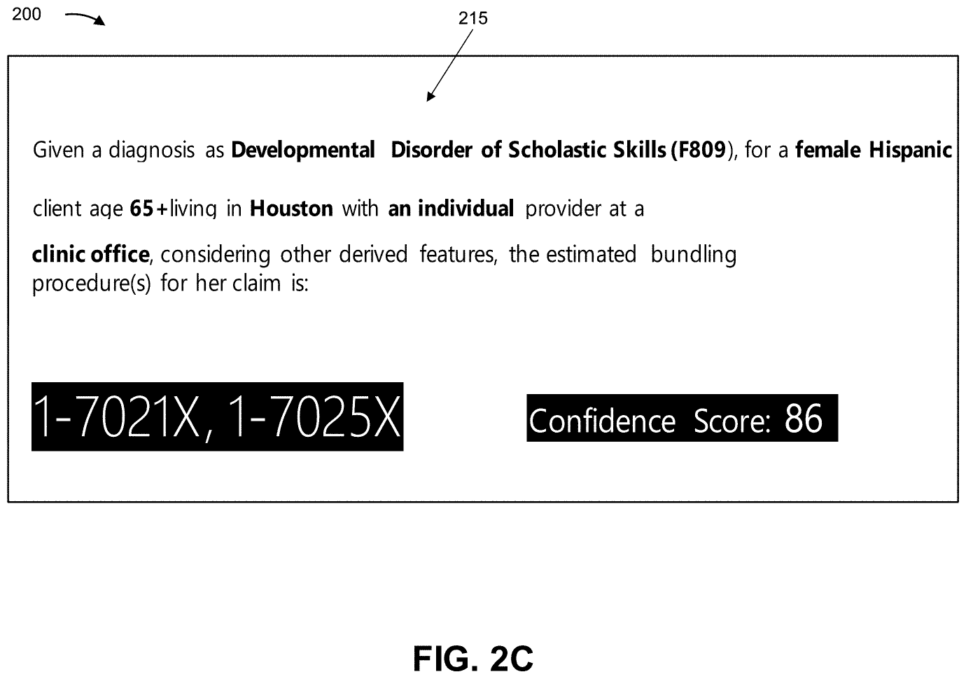

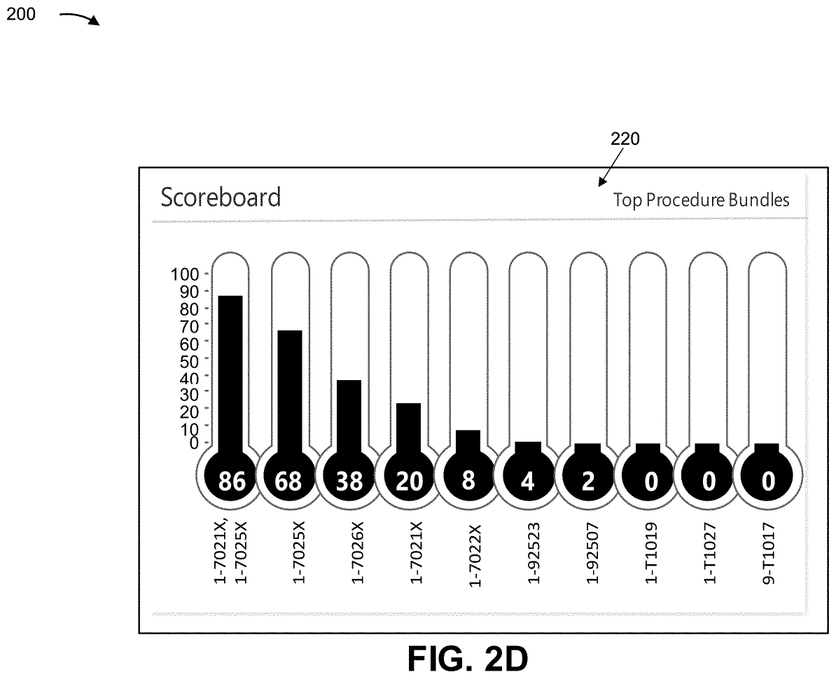

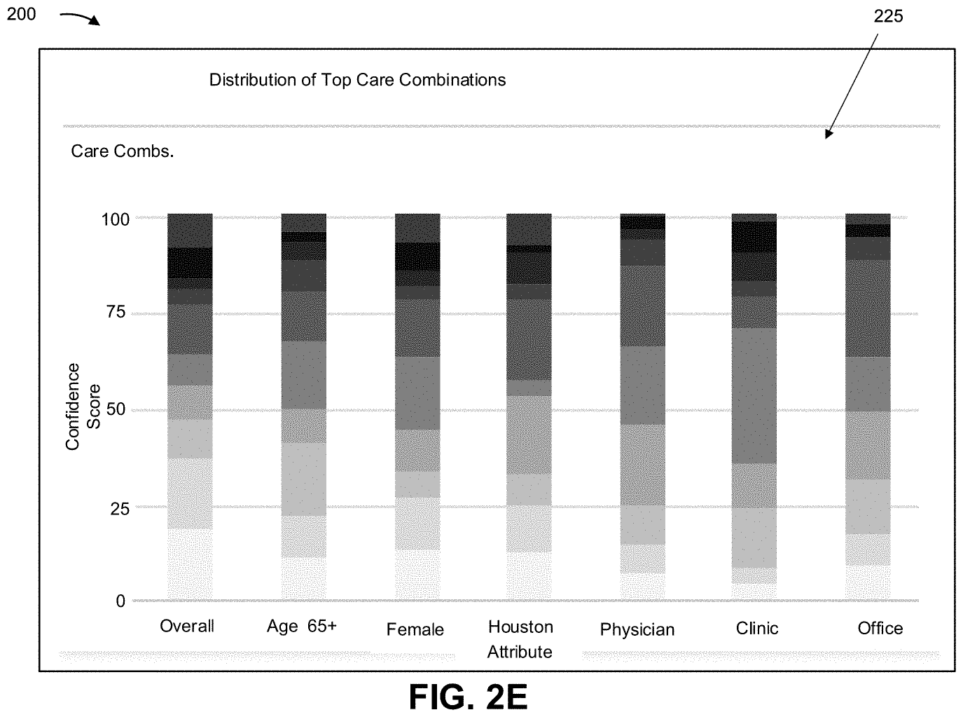

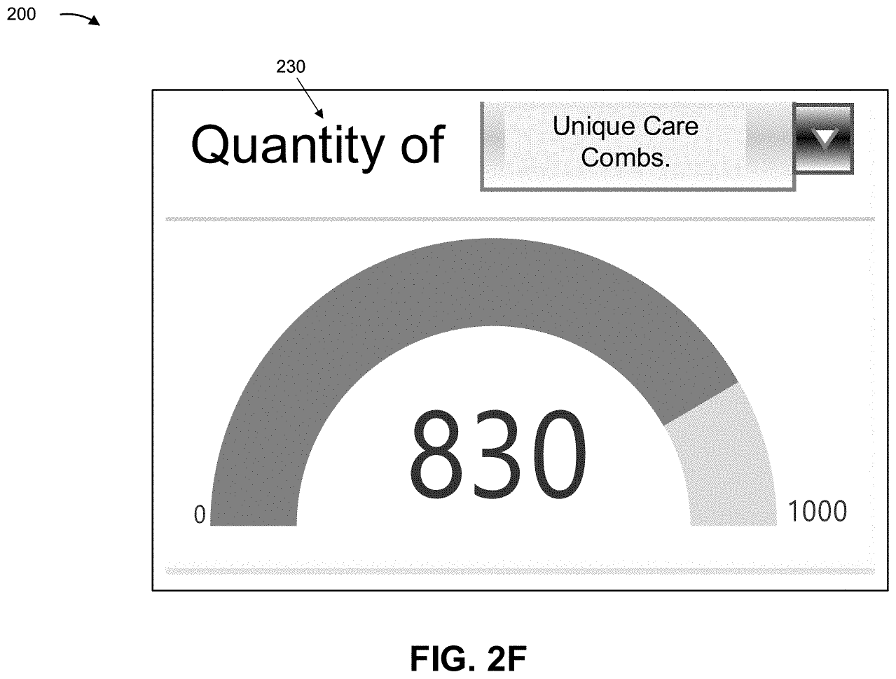

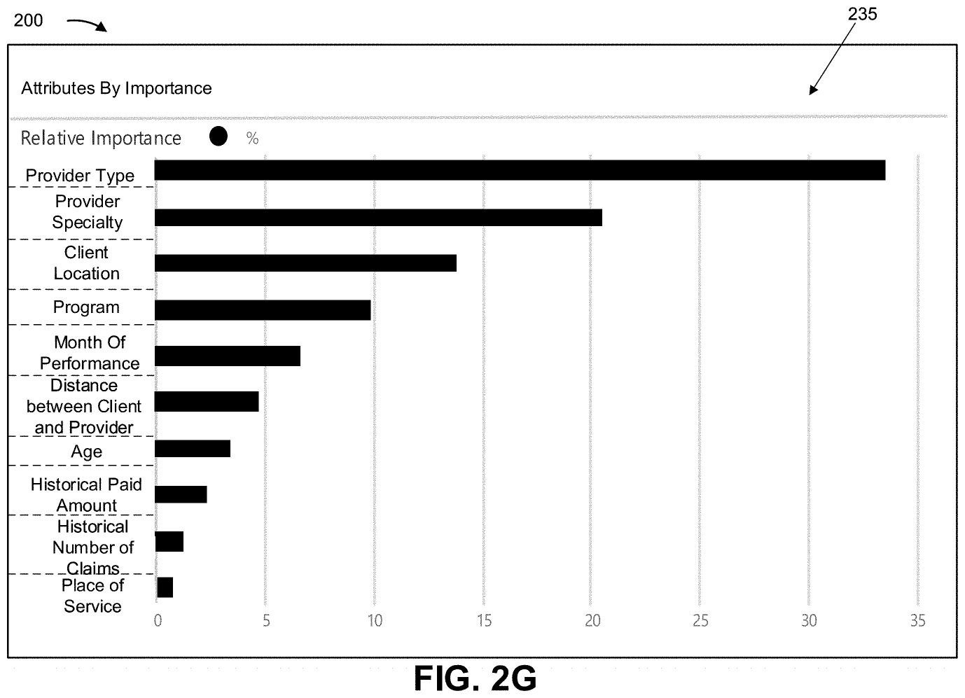

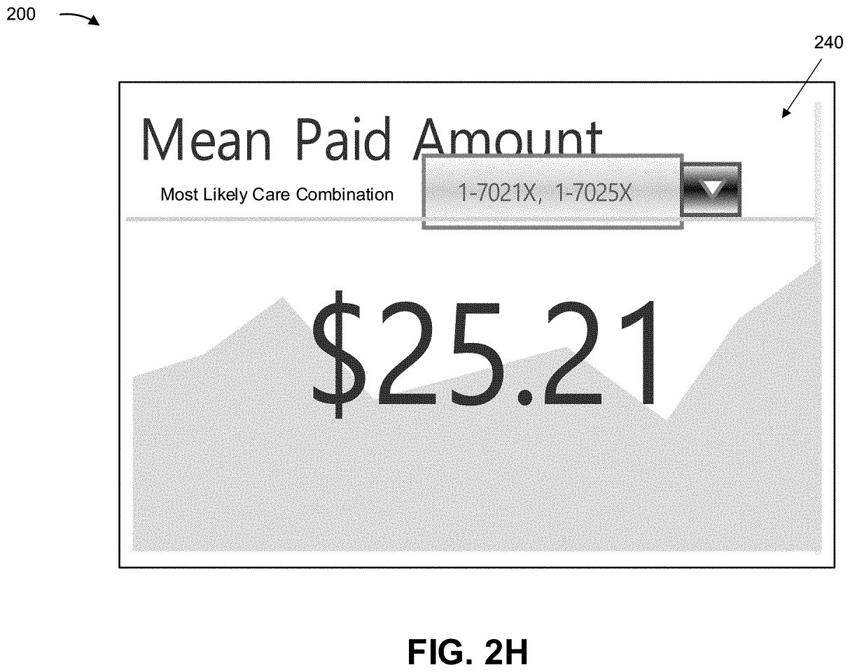

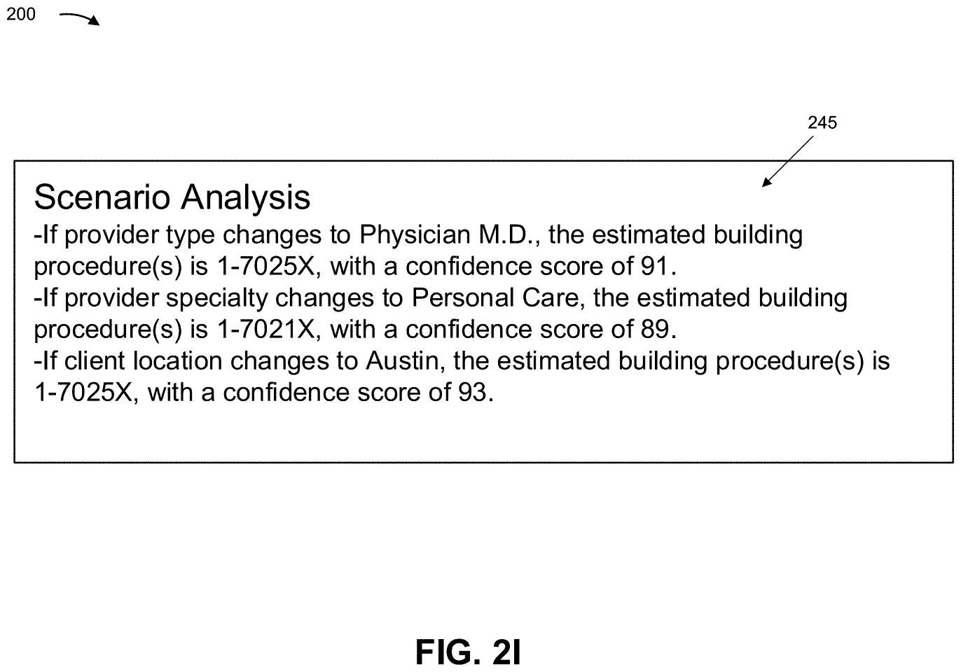

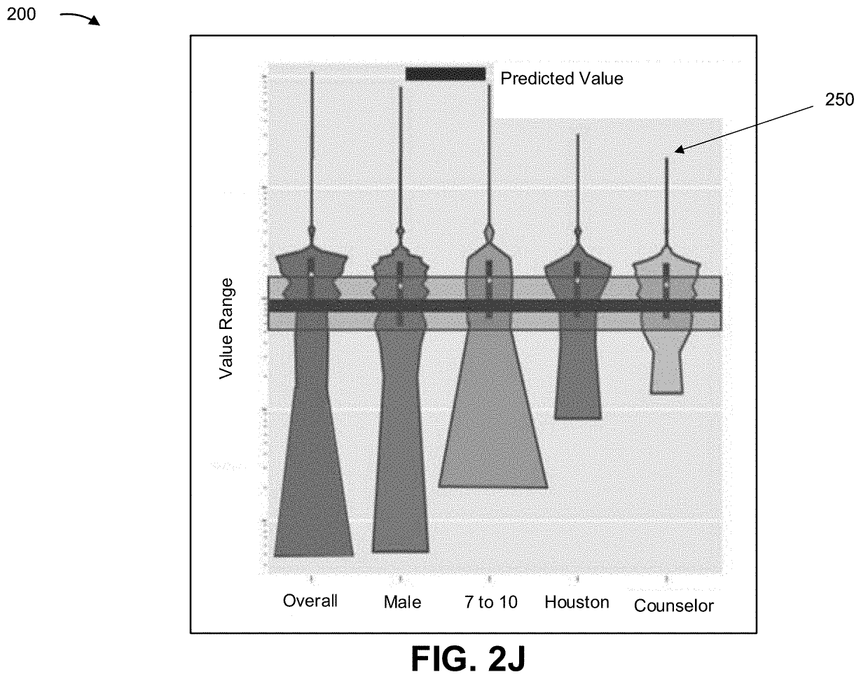

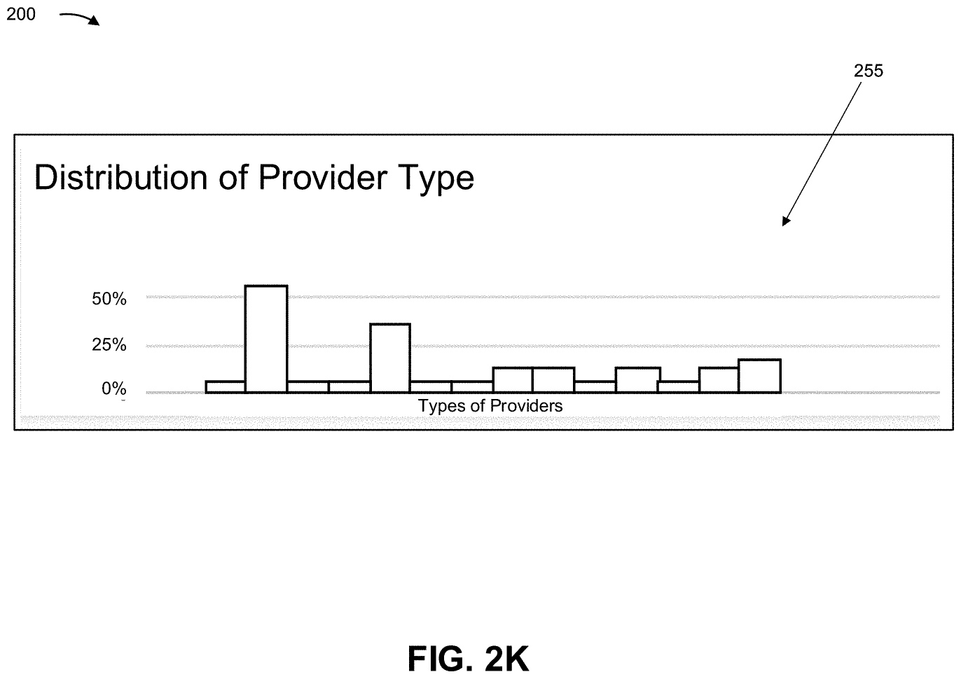

FIGS. 2A-2K are diagrams of one or more example implementations 200 described herein. FIGS. 2A-2K show examples of UIs (e.g., reporting UIs described elsewhere herein) that a predictive analysis platform may use to provide data, results of analyses, predictions, and/or the like for display.

As shown in FIG. 2A, and by reference number 205, the predictive analysis platform may provide a UI for display that includes information that identifies predictors for various diagnoses. For example, a user of the UI may select a diagnosis from a “Diagnosis” drop down UI element and/or a particular individual from a “ClientPCN#” drop down UI element, or may select values for various demographics associated with the individual, and the predictive analysis platform may predict care to be provided to the individual or an individual with the same values for the various demographics, a value of the care, and/or the like (e.g., based on user selection of an “Estimate” button described below and shown in FIG. 2B).

Turning to FIG. 2B, and as shown by reference number 210, the predictive analysis platform may provide a UI for display that includes information that identifies predictors for various providers. For example, a user of the UI may select a provider from a “Provider TPI #” drop down UI element, various attributes related to a provider that provided care to an individual to be analyzed, and/or the like, and the predictive analysis platform may use this information to perform an analysis, to generate a prediction, and/or the like, in the manner described herein (e.g., based on a user selection of an “Estimate” button on the UI).

Turning to FIG. 2C, and as shown by reference number 215, the predictive analysis platform may provide a UI for display that includes information that identifies a result of an analysis, a prediction that the predictive analysis platform generated, a score that the predictive analysis platform generated, and/or the like. For example, the UI may include information that identifies a diagnosis for an individual (e.g., shown as “Developmental Disorder of Scholastic Skills (F809)”), attributes of an individual that were the strongest relative factors in a result of an analysis, a score, a prediction, and/or the like that the predictive analysis platform generated, and/or the like (e.g., shown as “female,” “Hispanic,” “65+,” “Houston,” “individual provider,” and “clinic office”), a predicted (or recommended) combination of care to be provided to the individual (e.g., shown as “1-7021X, 1-7025X”), a score related to the predicted (or recommended) combination of care that indicates a confidence level associated with the predicted (or recommended) combination of care, and/or the like.

Turning to FIG. 2D, and as shown by reference number 220, the predictive analysis platform may provide a UI for display that includes information that identifies scores for various care that could be provided to the individual. For example, the predictive analysis platform may identify various care that could be provided to an individual, and may determine scores for the various care that indicate a confidence level that particular care is optimal for the individual based on attributes of the individual, a diagnosis, a provider, and/or the like.

Turning to FIG. 2E, and as shown by reference number 225, the predictive analysis platform may provide a UI for display that includes information that identifies various combinations of care for an individual by attribute of the individual. For example, the UI may include information that identifies recommended or predicted combinations of care by attribute of an individual (e.g., shown as “Overall,” “Age 65+,” “Female,” and so forth), where different colors shown with respect to each attribute identify different types of care or different combinations of care. Continuing with the previous example, the UI may be configured such that the predicted combinations of care for each attribute are organized by corresponding confidence scores (e.g., where a confidence score indicates a likelihood that particular care is to be provided to an individual or included in a combination of care provided to the individual). In some implementations, the predictive analysis platform may generate a recommended or predicted combination of care based on recommended or predicted combinations for each attribute (e.g., by averaging the combinations across the various attributes, by weighting the various attributes, by selecting care that is associated with a threshold confidence score across the various attributes, and/or the like).

Turning to FIG. 2F, and as shown by reference number 230, the predictive analysis platform may provide a UI for display that includes information that identifies a quantity of unique care combinations that the predictive analysis platform analyzed for an individual (e.g., out of a total quantity possible care combinations). Turning to FIG. 2G, and as shown by reference number 235, the predictive analysis platform may provide a UI for display that includes information that identifies attributes of an individual or a provider by importance. For example, an attribute for an individual or a provider may be weighted as more important relative to another attribute if the attribute had more of an impact on a predicted combination of care that the predictive analysis platform recommended or predicted to be provided to an individual.

Turning to FIG. 2H, and as shown by reference number 240, the predictive analysis platform may provide a UI for display that includes information that identifies a most likely care combination for an individual. For example, the UI may identify a combination of care that the predictive analysis platform recommended and/or predicted to be provided to an individual, a predicted cost of the combination of care, and/or the like. Turning to FIG. 2I, and as shown by reference number 245, the predictive analysis platform may provide a UI for display that includes information that identifies a result of a scenario analysis. For example, the predictive analysis platform may perform a scenario analysis as described herein, and the UI may include information that identifies a manner in which a score, predicted care (or recommended care), and/or the like may change based on changes to attributes of the individual, a provider, and/or the like.

Turning to FIG. 2J, and as shown by reference number 250, the predictive analysis platform may provide a UI for display that includes information that identifies a manner in which a predicted value for care predicted (or recommended) to be provided to an individual is determined by attributes of an individual. For example, the UI may include a range of predicted values for care to be provided to the individual by attribute of the individual and the predictive analysis platform may determine a predicted value of care by averaging the range of predicted values for different attributes, by weighting the range of predicted values, and/or the like (e.g., the predicted value is shown by the dark horizontal line across the ranges of predicted values in FIG. 2J). Turning to FIG. 2K, and as shown by reference number 255, the predictive analysis platform may provide a UI for display that includes information that identifies a distribution related to various types of providers. For example, the UI may include information that identifies a quantity of each of various types of providers associated with an analysis that the predictive analysis platform performed.

As indicated above, FIGS. 2A-2K are provided merely as one or more examples. Other examples may differ from what is described with regard to FIGS. 2A-2K.

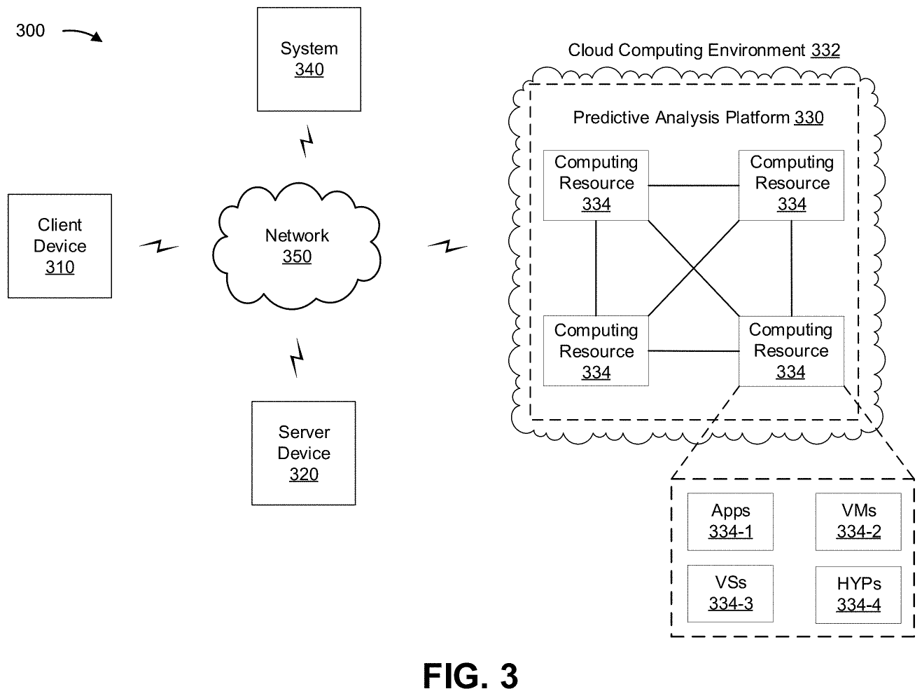

FIG. 3 is a diagram of an example environment 300 in which systems and/or methods described herein may be implemented. As shown in FIG. 3, environment 300 may include a client device 310, a server device 320, a predictive analysis platform 330 hosted within a cloud computing environment 332 that includes a set of computing resources 334, a system 340, and a network 350. Devices of environment 300 may interconnect via wired connections, wireless connections, or a combination of wired and wireless connections.

Client device 310 includes one or more devices capable of receiving, generating, storing, processing, and/or providing data described herein. For example, client device 310 may include a mobile phone (e.g., a smart phone, a radiotelephone, etc.), a laptop computer, a tablet computer, a handheld computer, a gaming device, a wearable communication device (e.g., a smart wristwatch, a pair of smart eyeglasses, etc.), a desktop computer, or a similar type of device. In some implementations, client device 310 may receive, from predictive analysis platform 330, a result of an analysis of data performed by predictive analysis platform 330, as described elsewhere herein.

Server device 320 includes one or more devices capable of receiving, generating storing, processing, and/or providing data described herein. For example, server device 320 may include a server (e.g., in a data center or a cloud computing environment), a data center (e.g., a multi-server micro datacenter), a workstation computer, a virtual machine (VIVI) provided in a cloud computing environment, or a similar type of device. In some implementations, server device 320 may include a communication interface that allows server device 320 to receive information from and/or transmit information to other devices in environment 300. In some implementations, server device 320 may be a physical device implemented within a housing, such as a chassis. In some implementations, server device 320 may be a virtual device implemented by one or more computer devices of a cloud computing environment or a data center. In some implementations, server device 320 may provide, to predictive analysis platform 330, data for processing by predictive analysis platform 330, as described elsewhere herein.

Predictive analysis platform 330 includes one or more devices capable of receiving, generating, storing, processing, and/or providing data described herein. For example, predictive analysis platform 330 may include a cloud server or a group of cloud servers. In some implementations, predictive analysis platform 330 may be designed to be modular such that certain software components can be swapped in or out depending on a particular need. As such, predictive analysis platform 330 may be easily and/or quickly reconfigured for different uses.

In some implementations, as shown in FIG. 3, predictive analysis platform 330 may be hosted in cloud computing environment 332. Notably, while implementations described herein describe predictive analysis platform 330 as being hosted in cloud computing environment 332, in some implementations, predictive analysis platform 330 may be non-cloud-based (i.e., may be implemented outside of a cloud computing environment) or may be partially cloud-based.

Cloud computing environment 332 includes an environment that hosts predictive analysis platform 330. Cloud computing environment 332 may provide computation, software, data access, storage, and/or other services that do not require end-user knowledge of a physical location and configuration of a system and/or a device that hosts predictive analysis platform 330. As shown, cloud computing environment 332 may include a group of computing resources 334 (referred to collectively as “computing resources 334” and individually as “computing resource 334”).

Computing resource 334 includes one or more personal computers, workstation computers, server devices, or another type of computation and/or communication device. In some implementations, computing resource 334 may host predictive analysis platform 330. The cloud resources may include compute instances executing in computing resource 334, storage devices provided in computing resource 334, data transfer devices provided by computing resource 334, etc. In some implementations, computing resource 334 may communicate with other computing resources 334 via wired connections, wireless connections, or a combination of wired and wireless connections.

As further shown in FIG. 3, computing resource 334 may include a group of cloud resources, such as one or more applications (“APPs”) 334-1, one or more virtual machines (“VMs”) 334-2, one or more virtualized storages (“VSs”) 334-3, or one or more hypervisors (“HYPs”) 334-4.

Application 334-1 includes one or more software applications that may be provided to or accessed by one or more devices of environment 300. Application 334-1 may eliminate a need to install and execute the software applications on devices of environment 300. For example, application 334-1 may include software associated with predictive analysis platform 330 and/or any other software capable of being provided via cloud computing environment 332. In some implementations, one application 334-1 may send/receive information to/from one or more other applications 334-1, via virtual machine 334-2. In some implementations, application 334-1 may include a software application associated with one or more databases and/or operating systems. For example, application 334-1 may include an enterprise application, a functional application, an analytics application, and/or the like.

Virtual machine 334-2 includes a software implementation of a machine (e.g., a computer) that executes programs like a physical machine. Virtual machine 334-2 may be either a system virtual machine or a process virtual machine, depending upon use and degree of correspondence to any real machine by virtual machine 334-2. A system virtual machine may provide a complete system platform that supports execution of a complete operating system (“OS”). A process virtual machine may execute a single program, and may support a single process. In some implementations, virtual machine 334-2 may execute on behalf of a user (e.g., a user of client device 310), and may manage infrastructure of cloud computing environment 332, such as data management, synchronization, or long-duration data transfers.

Virtualized storage 334-3 includes one or more storage systems and/or one or more devices that use virtualization techniques within the storage systems or devices of computing resource 334. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 334-4 provides hardware virtualization techniques that allow multiple operating systems (e.g., “guest operating systems”) to execute concurrently on a host computer, such as computing resource 334. Hypervisor 334-4 may present a virtual operating platform to the guest operating systems, and may manage the execution of the guest operating systems. Multiple instances of a variety of operating systems may share virtualized hardware resources.

System 340 includes one or more devices capable of receiving, generating, storing, processing, and/or providing data described herein. For example, system 340 may include a set of client device 310, a set of server device 320, and/or the like. In some implementations, system 340 may provide data, to predictive analysis platform 330, for analysis, as described elsewhere herein.

Network 350 includes one or more wired and/or wireless networks. For example, network 350 may include a cellular network (e.g., a long-term evolution (LTE) network, a code division multiple access (CDMA) network, a 3G network, a 4G network, a 5G network, another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, or the like, and/or a combination of these or other types of networks.

The number and arrangement of devices and networks shown in FIG. 3 are provided as one or more examples. In practice, there may be additional devices and/or networks, fewer devices and/or networks, different devices and/or networks, or differently arranged devices and/or networks than those shown in FIG. 3. Furthermore, two or more devices shown in FIG. 3 may be implemented within a single device, or a single device shown in FIG. 3 may be implemented as multiple, distributed devices. Additionally, or alternatively, a set of devices (e.g., one or more devices) of environment 300 may perform one or more functions described as being performed by another set of devices of environment 300.

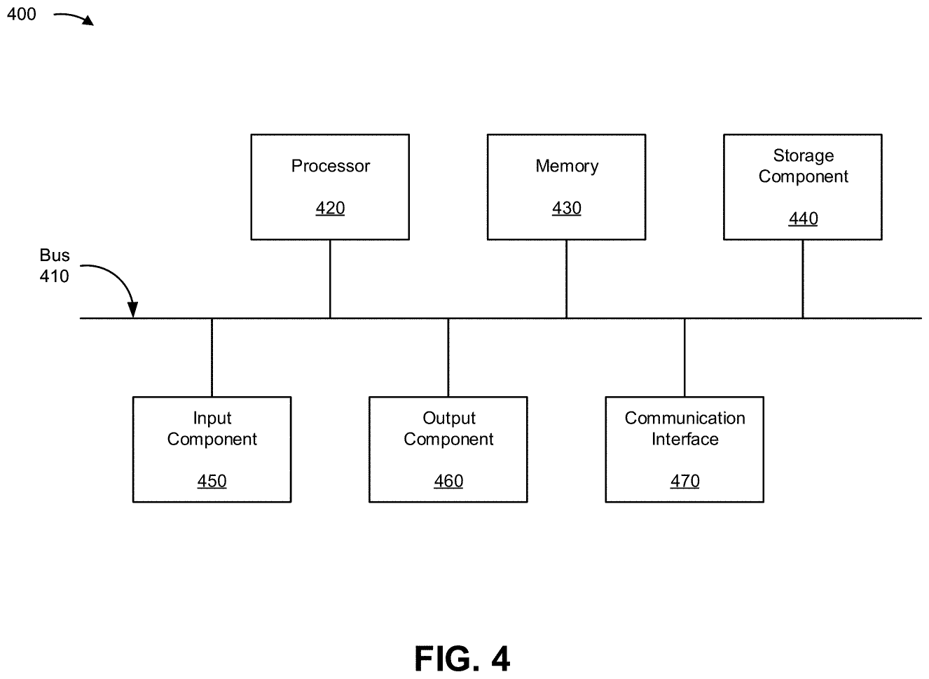

FIG. 4 is a diagram of example components of a device 400. Device 400 may correspond to client device 310, server device 320, predictive analysis platform 330, computing resource 334, and/or system 340. In some implementations, client device 310, server device 320, predictive analysis platform 330, computing resource 334, and/or system 340 may include one or more devices 400 and/or one or more components of device 400. As shown in FIG. 4, device 400 may include a bus 410, a processor 420, a memory 430, a storage component 440, an input component 450, an output component 460, and a communication interface 470.

Bus 410 includes a component that permits communication among multiple components of device 400. Processor 420 is implemented in hardware, firmware, and/or a combination of hardware and software. Processor 420 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 420 includes one or more processors capable of being programmed to perform a function. Memory 430 includes a random access memory (RAM), a read only memory (ROM), and/or another type of dynamic or static storage device (e.g., a flash memory, a magnetic memory, and/or an optical memory) that stores information and/or instructions for use by processor 420.

Storage component 440 stores information and/or software related to the operation and use of device 400. For example, storage component 440 may include a hard disk (e.g., a magnetic disk, an optical disk, and/or a magneto-optic disk), a solid state drive (SSD), a compact disc (CD), a digital versatile disc (DVD), a floppy disk, a cartridge, a magnetic tape, and/or another type of non-transitory computer-readable medium, along with a corresponding drive.

Input component 450 includes a component that permits device 400 to receive information, such as via user input (e.g., a touch screen display, a keyboard, a keypad, a mouse, a button, a switch, and/or a microphone). Additionally, or alternatively, input component 450 may include a component for determining location (e.g., a global positioning system (GPS) component) and/or a sensor (e.g., an accelerometer, a gyroscope, an actuator, another type of positional or environmental sensor, and/or the like). Output component 460 includes a component that provides output information from device 400 (via, e.g., a display, a speaker, a haptic feedback component, an audio or visual indicator, and/or the like).

Communication interface 470 includes a transceiver-like component (e.g., a transceiver, a separate receiver, a separate transmitter, and/or the like) that enables device 400 to communicate with other devices, such as via a wired connection, a wireless connection, or a combination of wired and wireless connections. Communication interface 470 may permit device 400 to receive information from another device and/or provide information to another device. For example, communication interface 470 may include an Ethernet interface, an optical interface, a coaxial interface, an infrared interface, a radio frequency (RF) interface, a universal serial bus (USB) interface, a Wi-Fi interface, a cellular network interface, and/or the like.

Device 400 may perform one or more processes described herein. Device 400 may perform these processes based on processor 420 executing software instructions stored by a non-transitory computer-readable medium, such as memory 430 and/or storage component 440. As used herein, the term “computer-readable medium” refers to a non-transitory memory device. A memory device includes memory space within a single physical storage device or memory space spread across multiple physical storage devices.

Software instructions may be read into memory 430 and/or storage component 440 from another computer-readable medium or from another device via communication interface 470. When executed, software instructions stored in memory 430 and/or storage component 440 may cause processor 420 to perform one or more processes described herein. Additionally, or alternatively, hardware circuitry may be used in place of or in combination with software instructions to perform one or more processes described herein. Thus, implementations described herein are not limited to any specific combination of hardware circuitry and software.

The number and arrangement of components shown in FIG. 4 are provided as an example. In practice, device 400 may include additional components, fewer components, different components, or differently arranged components than those shown in FIG. 4. Additionally, or alternatively, a set of components (e.g., one or more components) of device 400 may perform one or more functions described as being performed by another set of components of device 400.

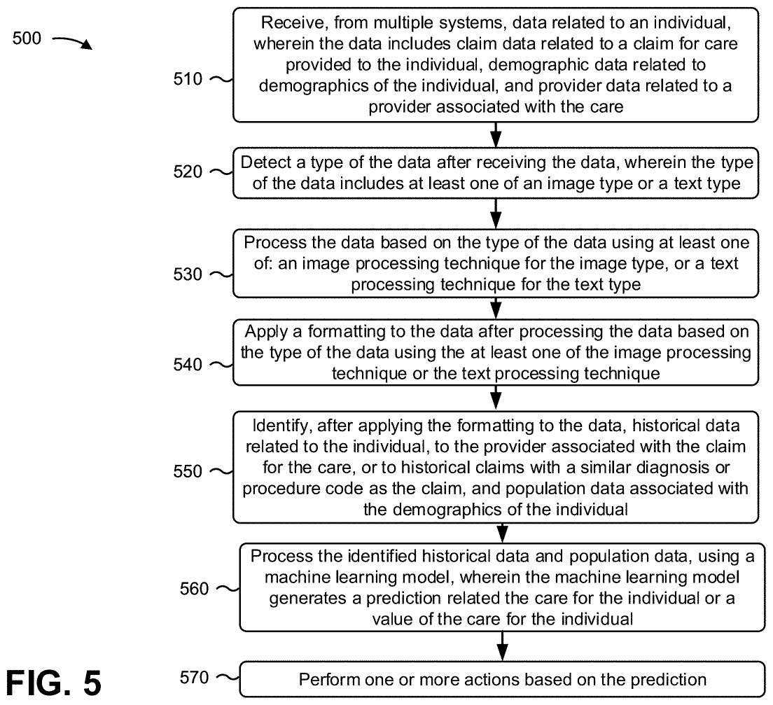

FIG. 5 is a flow chart of an example process 500 for performing predictive analysis. In some implementations, one or more process blocks of FIG. 5 may be performed by a predictive analysis platform (e.g., predictive analysis platform 330). In some implementations, one or more process blocks of FIG. 5 may be performed by another device or a group of devices separate from or including the predictive analysis platform, such as a client device (e.g., client device 310), a server device (e.g., server device 320), a computing resource (e.g., computing resource 334), and a system (e.g., system 340).

As shown in FIG. 5, process 500 may include receiving, from multiple systems, data related to an individual, wherein the data includes claim data related to a claim for care provided to the individual, demographic data related to demographics of the individual, and provider data related to a provider associated with the care (block 510). For example, the predictive analysis platform (e.g., using computing resource 334, processor 420, input component 450, communication interface 470, and/or the like) may receive, from multiple systems, data related to an individual, as described above. In some implementations, the data includes claim data related to a claim for care provided to the individual, demographic data related to demographics of the individual, and provider data related to a provider associated with the care.

As further shown in FIG. 5, process 500 may include detecting a type of the data after receiving the data, wherein the type of the data includes at least one of an image type or a text type (block 520). For example, the predictive analysis platform (e.g., using processor 420, and/or the like) may detect a type of the data after receiving the data, as described above. In some implementations, the type of the data includes at least one of an image type or a text type.

As further shown in FIG. 5, process 500 may include processing the data based on the type of the data using at least one of: an image processing technique for the image type, or a text processing technique for the text type (block 530). For example, the predictive analysis platform (e.g., using computing resource 334, processor 420, and/or the like) may process the data based on the type of the data using at least one of: an image processing technique for the image type, or a text processing technique for the text type, as described above.

As further shown in FIG. 5, process 500 may include applying a formatting to the data after processing the data based on the type of the data using the at least one of the image processing technique or the text processing technique (block 540). For example, the predictive analysis platform (e.g., using computing resource 334, processor 420, and/or the like) may apply a formatting to the data after processing the data based on the type of the data using the at least one of the image processing technique or the text processing technique, as described above.

As further shown in FIG. 5, process 500 may include identifying, after applying the formatting to the data, historical data related to the individual, to the provider associated with the claim for the care, or to historical claims with a similar diagnosis or procedure code as the claim, and population data associated with the demographics of the individual (block 550). For example, the predictive analysis platform (e.g., using computing resource 334, processor 420, and/or the like) may identify, after applying the formatting to the data, historical data related to the individual, to the provider associated with the claim for the care, or to historical claims with a similar diagnosis or procedure code as the claim, and population data associated with the demographics of the individual, as described above.

As further shown in FIG. 5, process 500 may include processing the identified historical data and population data, using a machine learning model, wherein the machine learning model generates a prediction related to the care for the individual or a value of the care for the individual (block 560). For example, the predictive analysis platform (e.g., using computing resource 334, processor 420, and/or the like) may process the identified historical data and population data, using a machine learning model, as described above. In some implementations, the machine learning model generates a prediction related to the care for the individual or a value of the care for the individual.

As further shown in FIG. 5, process 500 may include performing one or more actions based on the prediction (block 570). For example, the predictive analysis platform (e.g., using computing resource 334, processor 420, memory 430, storage component 440, output component 460, communication interface 470, and/or the like) may perform one or more actions based on the prediction, as described above.

Process 500 may include additional implementations, such as any single implementation or any combination of implementations described below and/or in connection with one or more other processes described elsewhere herein.

In some implementations, the predictive analysis platform may detect the type of the data based on a form of the data or a file extension of the data, wherein the form of the data or the file extension of the data indicates that the data is the image type or the text type. In some implementations, the predictive analysis platform may anonymize, after receiving the data, the data by replacing values of particular data elements of the data with anonymizing values.

In some implementations, the predictive analysis platform may process, from the data, information that identifies the individual using an anonymization technique to form an anonymized identifier, may perform a comparison of the anonymized identifier and multiple other anonymized identifiers in one or more data structures after processing the information to form the anonymized identifier, and may detect, based on a result of the comparison, a match between the anonymized identifier and the multiple other anonymized identifiers. In some implementations, the predictive analysis platform may select the at least one of the image processing technique or the text processing technique based on the type of the data, wherein the image processing technique is selected for the image type, or the text processing technique is selected for the text type, and may process the data using the at least one of the image processing technique or the text processing technique after selecting the at least one of the image processing technique or the text processing technique.

In some implementations, the predictive analysis platform may generate a score based on a result of processing the data using the machine learning model, wherein the score indicates a confidence level of the prediction, and may output, after generating the score, information that identifies the prediction and the score. In some implementations, the predictive analysis platform may perform, after identifying the historical data and the population data, an analysis of the data in a context of the historical data and the population data, wherein the analysis includes at least one of: a scenario analysis, a value analysis for the care, an analysis of a combination of care for the individual, or an analysis of a length of time for care to be provided to the individual, and may populate a set of user interface elements of a user interface with information that identifies a result of the analysis.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

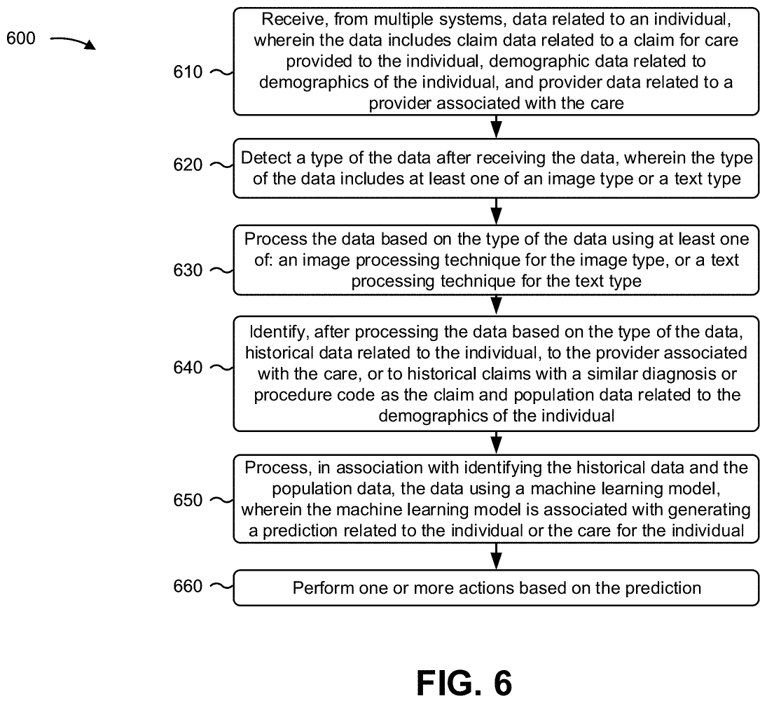

FIG. 6 is a flow chart of an example process 600 for performing predictive analysis. In some implementations, one or more process blocks of FIG. 6 may be performed by a predictive analysis platform (e.g., predictive analysis platform 330). In some implementations, one or more process blocks of FIG. 6 may be performed by another device or a group of devices separate from or including the predictive analysis platform, such as a client device (e.g., client device 310), a server device (e.g., server device 320), a computing resource (e.g., computing resource 334), and a system (e.g., system 340).

As shown in FIG. 6, process 600 may include receiving, from multiple systems, data related to an individual, wherein the data includes claim data related to a claim for care provided to the individual, demographic data related to demographics of the individual, and provider data related to a provider associated with the care (block 610). For example, the predictive analysis platform (e.g., using computing resource 334, processor 420, input component 450, communication interface 470, and/or the like) may receive, from multiple systems, data related to an individual, as described above. In some implementations, the data includes claim data related to a claim for care provided to the individual, demographic data related to demographics of the individual, and provider data related to a provider associated with the care.

As further shown in FIG. 6, process 600 may include detecting a type of the data after receiving the data, wherein the type of the data includes at least one of an image type or a text type (block 620). For example, the predictive analysis platform (e.g., using computing resource 334, processor 420, and/or the like) may detect a type of the data after receiving the data, as described above. In some implementations, the type of the data includes at least one of an image type or a text type.

As further shown in FIG. 6, process 600 may include processing the data based on the type of the data using at least one of: an image processing technique for the image type, or a text processing technique for the text type (block 630). For example, the predictive analysis platform (e.g., using computing resource 334, processor 420, and/or the like) may process the data based on the type of the data using at least one of: an image processing technique for the image type, or a text processing technique for the text type, as described above.

As further shown in FIG. 6, process 600 may include identifying, after processing the data based on the type of the data, historical data related to the individual, to the provider associated with the care, or to historical claims with a similar diagnosis or procedure code as the claim, and population data related to the demographics of the individual (block 640). For example, the predictive analysis platform (e.g., using computing resource 334, processor 420, and/or the like) may identify, after processing the data based on the type of the data, historical data related to the individual, to the provider associated with the care, or to historical claims with a similar diagnosis or procedure code as the claim, and population data related to the demographics of the individual, as described above.

As further shown in FIG. 6, process 600 may include processing, in association with identifying the historical data and the population data, the data using a machine learning model, wherein the machine learning model is associated with generating a prediction related to the individual or the care for the individual (block 650). For example, the predictive analysis platform (e.g., using computing resource 334, processor 420, and/or the like) may process, in association with identifying the historical data and the population data, the data using a machine learning model, as described above. In some implementations, the machine learning model is associated with generating a prediction related to the individual or the care for the individual.

As further shown in FIG. 6, process 600 may include performing one or more actions based on the prediction (block 660). For example, the predictive analysis platform (e.g., using computing resource 334, processor 420, memory 430, storage component 440, output component 460, communication interface 470, and/or the like) may perform one or more actions based on the prediction, as described above.

Process 600 may include additional implementations, such as any single implementation or any combination of implementations described below and/or in connection with one or more other processes described elsewhere herein.

In some implementations, the predictive analysis platform may generate a report related to the prediction after processing the data using the machine learning model, and may output the report for display after generating the report. In some implementations, the predictive analysis platform may perform an analysis of the prediction generated from the machine learning model, and may cause the claim to be approved or denied based on a result of the analysis, or may cause a value for the care to be adjusted based on the result of the analysis.

In some implementations, the predictive analysis platform may perform an analysis of the prediction generated from the machine learning model, and may generate a recommendation related to the care or a value of the care. In some implementations, the predictive analysis platform may perform an analysis of the data in a context of the historical data and the population data after identifying the historical data and the population data.

In some implementations, the predictive analysis platform may train the machine learning model using the historical data and the population data prior to processing the data using the machine learning model. In some implementations, the predictive analysis platform may receive the machine learning model prior to processing the data using the machine learning model.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

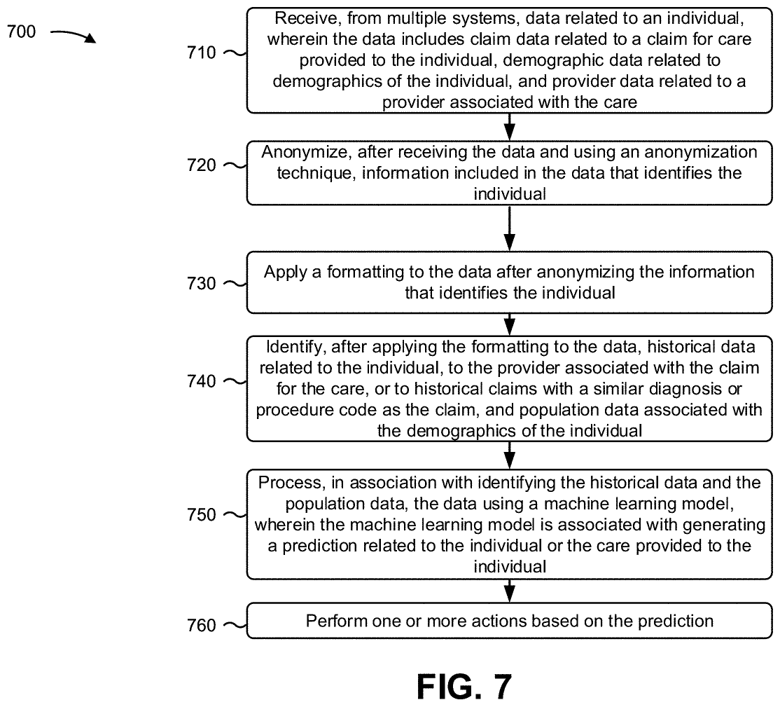

FIG. 7 is a flow chart of an example process 700 for performing predictive analysis. In some implementations, one or more process blocks of FIG. 7 may be performed by a predictive analysis platform (e.g., predictive analysis platform 330). In some implementations, one or more process blocks of FIG. 7 may be performed by another device or a group of devices separate from or including the predictive analysis platform, such as a client device (e.g., client device 310), a server device (e.g., server device 320), a computing resource (e.g., computing resource 334), and a system (e.g., system 340).

As shown in FIG. 7, process 700 may include receiving, from multiple systems, data related to an individual, wherein the data includes claim data related to a claim for care provided to the individual, demographic data related to demographics of the individual, and provider data related to a provider associated with the care (block 710). For example, the predictive analysis platform (e.g., using computing resource 334, processor 420, input component 450, communication interface 470, and/or the like) may receive, from multiple systems, data related to an individual, as described above. In some implementations, the data includes claim data related to a claim for care provided to the individual, demographic data related to demographics of the individual, and provider data related to a provider associated with the care.

As further shown in FIG. 7, process 700 may include anonymizing, after receiving the data and using an anonymization technique, information included in the data that identifies the individual (block 720). For example, the predictive analysis platform (e.g., using computing resource 334, processor 420, and/or the like) may anonymize, after receiving the data and using an anonymization technique, information included in the data that identifies the individual, as described above.

As further shown in FIG. 7, process 700 may include applying a formatting to the data after anonymizing the information that identifies the individual (block 730). For example, the predictive analysis platform (e.g., using computing resource 334, processor 420, and/or the like) may apply a formatting to the data after anonymizing the information that identifies the individual, as described above.

As further shown in FIG. 7, process 700 may include identifying, after applying the formatting to the data, historical data related to the individual, to the provider associated with the claim for the care, to historical claims with a similar diagnosis or procedure code as the claim, and population data associated with the demographics of the individual (block 740). For example, the predictive analysis platform (e.g., using computing resource 334, processor 420, and/or the like) may identify, after applying the formatting to the data, historical data related to the individual, to the provider associated with the claim for the care, to historical claims with a similar diagnosis or procedure code as the claim, and population data associated with the demographics of the individual, as described above.

As further shown in FIG. 7, process 700 may include processing, in association with identifying the historical data and the population data, the data using a machine learning model, wherein the machine learning model is associated with generating a prediction related to the individual or the care provided to the individual (block 750). For example, the predictive analysis platform (e.g., using computing resource 334, processor 420, and/or the like) may process, in association with identifying the historical data and the population data, the data using a machine learning model, as described above. In some implementations, the machine learning model is associated with generating a prediction related to the individual or the care provided to the individual.

As further shown in FIG. 7, process 700 may include performing one or more actions based on the prediction (block 760). For example, the predictive analysis platform (e.g., using computing resource 334, processor 420, memory 430, storage component 440, output component 460, communication interface, and/or the like) may perform one or more actions based on the prediction, as described above.

Process 700 may include additional implementations, such as any single implementation or any combination of implementations described below and/or in connection with one or more other processes described elsewhere herein.

In some implementations, the predictive analysis platform may detect the type of the data based on a form of the data or a file extension of the data, wherein the form of the data or the file extension of the data indicates that the data is an image type or a text type. In some implementations, the predictive analysis platform may detect a type of the data after receiving the data, and may process, based on the type of the data, the data using at least one of: an image processing technique, or a text processing technique.

In some implementations, the predictive analysis platform may select the at least one of the image processing technique or the text processing technique based on the type of the data, wherein the image processing technique is selected for an image type, or the text processing technique is selected for a text type, and may process the data using the at least one of the image processing technique or the text processing technique after selecting the at least one of the image processing technique or the text processing technique. In some implementations, the predictive analysis platform may generate a score based on a result of processing the data using the machine learning model, wherein the score indicates a similarity between the data and the historical data or between the data and the population data, and may generate, after generating the score, the prediction based on the score. In some implementations, the prediction is related to at least one of: the future care to be provided to the individual, a value of the future care, or a likelihood that the claim is a legitimate claim.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

The foregoing disclosure provides illustration and description, but is not intended to be exhaustive or to limit the implementations to the precise forms disclosed. Modifications and variations may be made in light of the above disclosure or may be acquired from practice of the implementations.

As used herein, the term “component” is intended to be broadly construed as hardware, firmware, and/or a combination of hardware and software.

Some implementations are described herein in connection with thresholds. As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Certain user interfaces have been described herein and/or shown in the figures. A user interface may include a graphical user interface, a non-graphical user interface, a text-based user interface, and/or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods described herein may be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method, comprising:
receiving, by a device and from multiple systems, data related to an individual,
wherein the data includes claim data related to a claim for care provided to the individual, demographic data related to demographics of the individual, and provider data related to a provider associated with the care;
detecting, by the device, a type of the data after receiving the data,
wherein the type of the data includes at least one of an image type or a text type;
processing, by the device, the data based on the type of the data using at least one of:
an image processing technique for the image type, or
a text processing technique for the text type;
applying, by the device, a formatting to the data after processing the data based on the type of the data using the at least one of the image processing technique or the text processing technique;
identifying, by the device and after applying the formatting to the data, historical data related to the individual, or to the provider associated with the claim for the care, and population data associated with the demographics of the individual;
processing, by the device, the identified historical data and population data, using a machine learning model,
wherein the machine learning model generates a prediction related to the care for the individual or a value of the care for the individual; and
performing, by the device, one or more actions based on the prediction.

2. The method of claim 1, wherein detecting the type of the data comprises:
detecting the type of the data based on a form of the data or a file extension of the data,
wherein the form of the data or the file extension of the data indicates that the data is the image type or the text type.

3. The method of claim 1, further comprising:
anonymizing, after receiving the data, the data by replacing values of particular data elements of the data with anonymizing values.

4. The method of claim 1, further comprising:
processing, from the data, information that identifies the individual using an anonymization technique to form an anonymized identifier; and
wherein identifying the historical data and the population data comprises:
performing a comparison of the anonymized identifier and multiple other anonymized identifiers in one or more data structures after processing the information to form the anonymized identifier; and
detecting, based on a result of the comparison, a match between the anonymized identifier and the multiple other anonymized identifiers.

5. The method of claim 1, further comprising:
selecting the at least one of the image processing technique or the text processing technique based on the type of the data,
wherein the image processing technique is selected for the image type, or the text processing technique is selected for the text type; and
wherein processing the data comprises:
processing the data using the at least one of the image processing technique or the text processing technique after selecting the at least one of the image processing technique or the text processing technique.

6. The method of claim 1, further comprising:
generating a score based on a result of processing the data using the machine learning model,
wherein the score indicates a confidence level of the prediction; and
outputting, after generating the score, information that identifies the prediction and the score.

7. The method of claim 1, wherein performing the one or more actions comprises:
performing, after identifying the historical data and the population data, an analysis of the data in a context of the historical data and the population data,
wherein the analysis includes at least one of:
a scenario analysis,
a value analysis for the care,
an analysis of a combination of care for the individual, or
an analysis of a length of time for care to be provided to the individual; and
populating a set of user interface elements of a user interface with information that identifies a result of the analysis.

8. A device, comprising:
one or more memories; and
one or more processors communicatively coupled to the one or more memories, to:
receive, from multiple systems, data related to an individual,
wherein the data includes claim data related to a claim for care provided to the individual, demographic data related to demographics of the individual, and provider data related to a provider associated with the care;
detect a type of the data after receiving the data,
wherein the type of the data includes at least one of an image type or a text type;
process the data based on the type of the data using at least one of:
an image processing technique for the image type, or
a text processing technique for the text type;
identify, after processing the data based on the type of the data, historical data related to the individual, to the provider associated with the care, or to historical claims with a similar diagnosis or procedure code as the claim, and population data related to the demographics of the individual;
process, in association with identifying the historical data and the population data, the data using a machine learning model,
wherein the machine learning model is associated with generating a
prediction related to the individual or the care for the individual; and
perform one or more actions based on the prediction.

9. The device of claim 8, wherein the one or more processors, when performing the one or more actions, are to:
generate a report related to the prediction after processing the data using the machine learning model; and
output the report for display after generating the report.

10. The device of claim 8, wherein the one or more processors, when performing the one or more actions, are to:
perform an analysis of the prediction generated from the machine learning model; and
cause the claim to be approved or denied based on a result of the analysis, or
cause a value for the care to be adjusted based on the result of the analysis.

11. The device of claim 8, wherein the one or more processors, when performing the one or more actions, are to:
perform an analysis of the prediction generated from the machine learning model; and
generate a recommendation related to the care or a value of the care.

12. The device of claim 8, wherein the one or more processors are further to:
perform an analysis of the data in a context of the historical data and the population data after identifying the historical data and the population data.

13. The device of claim 8, wherein the one or more processors are further to:
train the machine learning model using the historical data and the population data prior to processing the data using the machine learning model.

14. The device of claim 8, wherein the one or more processors are further to:
receive the machine learning model prior to processing the data using the machine learning model.

15. A non-transitory computer-readable medium storing instructions, the instructions comprising:
one or more instructions that, when executed by one or more processors of a device, cause the one or more processors to:
receive, from multiple systems, data related to an individual,
wherein the data includes claim data related to a claim for care provided to the individual, demographic data related to demographics of the individual, and provider data related to a provider associated with the care;
anonymize, after receiving the data and using an anonymization technique, information included in the data that identifies the individual;
apply a formatting to the data after anonymizing the information that identifies the individual;
identify, after applying the formatting to the data, historical data related to the individual, to the provider associated with the claim for the care, or to historical claims with a similar diagnosis or procedure code as the claim, and population data associated with the demographics of the individual;
process, in association with identifying the historical data and the population data, the data using a machine learning model,
wherein the machine learning model is associated with generating a prediction related to the individual or the care provided to the individual; and
perform one or more actions based on the prediction.

16. The non-transitory computer-readable medium of claim 15, wherein the one or more instructions, that cause the one or more processors to detect a type of the data, cause the one or more processors to:
detect the type of the data based on a form of the data or a file extension of the data,
wherein the form of the data or the file extension of the data indicates that the data is an image type or a text type.

17. The non-transitory computer-readable medium of claim 15, wherein the one or more instructions, when executed by the one or more processors, further cause the one or more processors to:
detect a type of the data after receiving the data; and
process, based on the type of the data, the data using at least one of:
an image processing technique, or
a text processing technique.

18. The non-transitory computer-readable medium of claim 17, wherein the one or more instructions, when executed by the one or more processors, further cause the one or more processors to:
select the at least one of the image processing technique or the text processing technique based on the type of the data,
wherein the image processing technique is selected for an image type, or the text processing technique is selected for a text type; and
wherein the one or more instructions, that cause the one or more processors to process the data using the at least one of the image processing technique or the text processing technique, cause the one or more processors to:
process the data using the at least one of the image processing technique or the text processing technique after selecting the at least one of the image processing technique or the text processing technique.

19. The non-transitory computer-readable medium of claim 15, wherein the one or more instructions, when executed by the one or more processors, further cause the one or more processors to:
generate a score based on a result of processing the data using the machine learning model,
wherein the score indicates a similarity between the data and the historical data or between the data and the population data; and
generate, after generating the score, the prediction based on the score.

20. The non-transitory computer-readable medium of claim 15, wherein the prediction is related to at least one of:
future care to be provided to the individual,
a value of the future care, or
a likelihood that the claim is a legitimate claim.
